SEQUENCE LISTING (117 sequences)

SEQ ID NO: 1 - DNA, 50 bp, Artificial Sequence
  DNA oligonucleotide
agcgaacgtggattttgtccgacatcggcaagctccctttttcgactatt

SEQ ID NO: 2 - DNA, 50 bp, Artificial Sequence
  DNA oligonucleotide
ccgatgtcggacttttacacgatcttcgcctgctgggttttgggagcttg

SEQ ID NO: 3 - DNA, 50 bp, Artificial Sequence
  DNA oligonucleotide
cgaagatcgtgtttttccacagttgattgcccttcacttttcccagcagg

SEQ ID NO: 4 - DNA, 50 bp, Artificial Sequence
  DNA oligonucleotide
aatcaactgtggtttttctcactggtgattagaatgcttttgtgaagggc

SEQ ID NO: 5 - DNA, 50 bp, Artificial Sequence
  DNA oligonucleotide
tcaccagtgagatttttgtcgtaccaggtgcatggatttttgcattctaa

SEQ ID NO: 6 - DNA, 50 bp, Artificial Sequence
  DNA oligonucleotide
cctggtacgacatttttccacgttcgctaatagtcgattttatccatgca

SEQ ID NO: 7 - RNA, 720 nt, Aequorea victoria
  CDS (1)..(720): eGFP sequence
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaa

SEQ ID NO: 8 - protein, 239 aa, Aequorea victoria
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 9 - DNA, 38 bp, Artificial Sequence
  DNA oligonucleotide
accaccccggtgaacagctcctcgcccttgctcaccat

SEQ ID NO: 10 - DNA, 35 bp, Artificial Sequence
  DNA oligonucleotide
cgatgcccttcagctcgatgcggttcaccagggtg

SEQ ID NO: 11 - DNA, 35 bp, Artificial Sequence
  DNA oligonucleotide
cgtttacgtcgccgtccagctcgaccaggatgggc

SEQ ID NO: 12 - DNA, 35 bp, Artificial Sequence
  DNA oligonucleotide
gccctcgccctcgccggacacgctgaacttgtggc

SEQ ID NO: 13 - DNA, 35 bp, Artificial Sequence
  DNA oligonucleotide
cttgtgccccaggatgttgccgtcctccttgaagt

SEQ ID NO: 14 - DNA, 35 bp, Artificial Sequence
  DNA oligonucleotide
ccagcaggaccatgtgatcgcgcttctcgttgggg

SEQ ID NO: 15 - DNA, 35 bp, Artificial Sequence
  DNA oligonucleotide
tctttgctcagggcggacttgggtgctcaggtagt

SEQ ID NO: 16 - DNA, 35 bp, Artificial Sequence
  DNA oligonucleotide
tgggccagggcacgggcagcttgccggtggtgcag

SEQ ID NO: 17 - DNA, 35 bp, Artificial Sequence
  DNA oligonucleotide
tcaccttgatgccgttcttctgcttgtcggccatg

SEQ ID NO: 18 - DNA, 35 bp, Artificial Sequence
  DNA oligonucleotide
gctgccgtcctcgatgttgtggcggatcttgaagt

SEQ ID NO: 19 - DNA, 35 bp, Artificial Sequence
  DNA oligonucleotide
gcactgcacgccgtaggtcagggtggtcacgaggg

SEQ ID NO: 20 - DNA, 35 bp, Artificial Sequence
  DNA oligonucleotide
atatagacgttgtggctgttgtagttgtactccag

SEQ ID NO: 21 - DNA, 35 bp, Artificial Sequence
  DNA oligonucleotide
atgaacttcagggtcagcttgccgtaggtggcatc

SEQ ID NO: 22 - DNA, 35 bp, Artificial Sequence
  DNA Oligonucleotide
tcgtgctgcttcatgtggtcggggtagcggctgaa

SEQ ID NO: 23 - DNA, 35 bp, Artificial Sequence
  DNA Oligonucleotide
ggggtgttctgctggtagtggtcggcgagctgcac

SEQ ID NO: 24 - DNA, 35 bp, Artificial Sequence
  DNA Oligonucleotide
ggttgtcgggcagcagcacggggccgtcgccgatg

SEQ ID NO: 25 - DNA, 35 bp, Artificial Sequence
  DNA Oligonucleotide
catgccgagagtgatcccggcggcggtcacgaact

SEQ ID NO: 26 - DNA, 35 bp, Artificial Sequence
  DNA Oligonucleotide
ggacgtagccttcgggcatggcggacttgaagaag

SEQ ID NO: 27 - DNA, 18 bp, Artificial Sequence
  DNA Oligonucleotide
ttacttgtacagctcgtc

SEQ ID NO: 28 - DNA, 35 bp, Artificial Sequence
  DNA Oligonucleotide
tcgccctcgaacttcacctcggcgcgggtcttgta

SEQ ID NO: 29 - DNA, 35 bp, Artificial Sequence
  DNA Oligonucleotide
gttgccgtcgtccttgaagaagatggtgcgctcct

SEQ ID NO: 30 - DNA, 27 bp, Artificial Sequence
  DNA Oligonucleotide
tttgatgaacttcagccagctcgattt

SEQ ID NO: 31 - DNA, 38 bp, Artificial Sequence
  DNA Oligonucleotide
ttttctcagggcggacttggctcgttgttttttttttt

SEQ ID NO: 32 - DNA, 35 bp, Artificial Sequence
  DNA Oligonucleotide
ttttctgaactatcgccctcgcccttttttttttt

SEQ ID NO: 33 - DNA, 43 bp, Artificial Sequence
  DNA Oligonucleotide
tttccaggatgggcaccactcgccgtggtcagcttgccgtttt

SEQ ID NO: 34 - DNA, 19 bp, Artificial Sequence
  DNA Oligonucleotide
ttttgtgggtctttgtttt

SEQ ID NO: 35 - DNA, 35 bp, Artificial Sequence
  DNA Oligonucleotide
tttggcagcacacgctgccgtcctggccatgtttt

SEQ ID NO: 36 - DNA, 35 bp, Artificial Sequence
  DNA Oligonucleotide
ttttttttttcgttctgctggtagtggtcggtttt

SEQ ID NO: 37 - DNA, 38 bp, Artificial Sequence
  DNA Oligonucleotide
ttttcatgtggtcggggtaaagaagtcgtgctgctttt

SEQ ID NO: 38 - DNA, 35 bp, Artificial Sequence
  DNA Oligonucleotide
tttatatagacgttgtgcgagctgttttttttttt

SEQ ID NO: 39 - DNA, 49 bp, Artificial Sequence
  DNA Oligonucleotide
ttttttttttttttttcttgctcgctcctggacgtagtttttttttttt

SEQ ID NO: 40 - DNA, 35 bp, Artificial Sequence
  DNA Oligonucleotide
tttttttttatggtgcaccatagtgatcccttttt

SEQ ID NO: 41 - DNA, 35 bp, Artificial Sequence
  DNA Oligonucleotide
ttttgcggttttcttctgcttgtccgatgtttttt

SEQ ID NO: 42 - DNA, 35 bp, Artificial Sequence
  DNA Oligonucleotide
tttttttttttaggtggctgtggccgtttacgttt

SEQ ID NO: 43 - DNA, 29 bp, Artificial Sequence
  DNA Oligonucleotide
ttttaccttgatgccgcaccagggttttt

SEQ ID NO: 44 - DNA, 47 bp, Artificial Sequence
  DNA oligonucleotide
ttttgcatggcggacttggcggctggcagcttgccggtggtgcattt

SEQ ID NO: 45 - DNA, 35 bp, Artificial Sequence
  DNA oligonucleotide
ttttaagcactgcacgccccttcggtttttttttt

SEQ ID NO: 46 - DNA, 26 bp, Artificial Sequence
  DNA oligonucleotide
tttgtggcggatcttgaagttctttt

SEQ ID NO: 47 - DNA, 35 bp, Artificial Sequence
  DNA oligonucleotide
ttttttttttacctcggatgtgatcgcgctttttt

SEQ ID NO: 48 - DNA, 35 bp, Artificial Sequence
  DNA oligonucleotide
tttgccgtcctccttgattgccgtttttttttttt

SEQ ID NO: 49 - DNA, 35 bp, Artificial Sequence
  DNA oligonucleotide
tttggcggcgcgtccttgaagaagggatgtttttt

SEQ ID NO: 50 - DNA, 35 bp, Artificial Sequence
  DNA oligonucleotide
ttttttttttcaggacccgcgggtcttgtagtttt

SEQ ID NO: 51 - DNA, 32 bp, Artificial Sequence
  DNA oligonucleotide
accaccccgaacttgtggccgtttccgtaggt

SEQ ID NO: 52 - DNA, 32 bp, Artificial Sequence
  DNA oligonucleotide
tggtcacgatgtggtcggggtagccttcgggc

SEQ ID NO: 53 - DNA, 32 bp, Artificial Sequence
  DNA oligonucleotide
gccgtagatgaacttcacgatggtcctgggtc

SEQ ID NO: 54 - DNA, 23 bp, Artificial Sequence
  DNA oligonucleotide
cttcacctcggcgcgggtctttt

SEQ ID NO: 55 - DNA, 18 bp, Artificial Sequence
  DNA oligonucleotide
ttttgtcggtagtgtttt

SEQ ID NO: 56 - DNA, 30 bp, Artificial Sequence
  DNA oligonucleotide
ttttcgtgctgcttcagggtgggccatttt

SEQ ID NO: 57 - DNA, 32 bp, Artificial Sequence
  DNA oligonucleotide
tcttctgctgatatagacgttgtgttgtactc

SEQ ID NO: 58 - DNA, 30 bp, Artificial Sequence
  DNA oligonucleotide
ttttccggacacgctggtgaacagcttttt

SEQ ID NO: 59 - DNA, 18 bp, Artificial Sequence
  DNA oligonucleotide
ttttaccttagttctttt

SEQ ID NO: 60 - DNA, 47 bp, Artificial Sequence
  DNA oligonucleotide
agcttgacgtcgccgtcccgatcgtcgcagctcgaccactttgctca

SEQ ID NO: 61 - DNA, 32 bp, Artificial Sequence
  DNA oligonucleotide
atggcggagtcgtccttgaagaagccctcgaa

SEQ ID NO: 62 - DNA, 18 bp, Artificial Sequence
  DNA oligonucleotide
tttttgtaggctgttttt

SEQ ID NO: 63 - DNA, 39 bp, Artificial Sequence
  DNA oligonucleotide
ttttcctcgcccttgctcaccatgttggggtggatgggc

SEQ ID NO: 64 - DNA, 50 bp, Artificial Sequence
  DNA oligonucleotide
tcgatgcggttcaccagggtgtcgatggtgcgctcaggatgccccctgga

SEQ ID NO: 65 - DNA, 32 bp, Artificial Sequence
  DNA oligonucleotide
acgctgccttgtggcggatcttgagatgccgt

SEQ ID NO: 66 - DNA, 31 bp, Artificial Sequence
  DNA oligonucleotide
gggcggacttgggtgctcaggtaggcagcag

SEQ ID NO: 67 - DNA, 32 bp, Artificial Sequence
  DNA oligonucleotide
cgtagcggctgaagcacggccattgtctgcac

SEQ ID NO: 68 - DNA, 30 bp, Artificial Sequence
  DNA oligonucleotide
ttttttgtagttgcccttgaagaagttttt

SEQ ID NO: 69 - DNA, 18 bp, Artificial Sequence
  DNA oligonucleotide
ttttgatgcaagtctttt

SEQ ID NO: 70 - DNA, 32 bp, Artificial Sequence
  DNA oligonucleotide
ggcatcgcgcttgccggtggtgcaggtcaggg

SEQ ID NO: 71 - DNA, 32 bp, Artificial Sequence
  DNA oligonucleotide
cagcttgtgttgccgtcctccttgccttcagc

SEQ ID NO: 72 - DNA, 30 bp, Artificial Sequence
  DNA oligonucleotide
ttttgggcacgggcacctcgccctcgtttt

SEQ ID NO: 73 - DNA, 32 bp, Artificial Sequence
  DNA oligonucleotide
cacggggcgggggtgttctgctggcgagctgc

SEQ ID NO: 74 - DNA, 18 bp, Artificial Sequence
  DNA oligonucleotide
tttttgtcggtggttttt

SEQ ID NO: 75 - DNA, 46 bp, Artificial Sequence
  DNA oligonucleotide
tgctcaggacagcggtgagcgggccgtttgttctgcttcgatgttg

SEQ ID NO: 76 - DNA, 47 bp, Artificial Sequence
  DNA oligonucleotide
tttcttgatgccgttcgtccagctaagtcgatcttgctcaccatttt

SEQ ID NO: 77 - DNA, 38 bp, Artificial Sequence
  DNA oligonucleotide
ttgtactccagccggttcacgaacttgtgacttgggtg

SEQ ID NO: 78 - DNA, 38 bp, Artificial Sequence
  DNA oligonucleotide
tttccaggatgggcaccacccctcctcgccgcccttca

SEQ ID NO: 79 - DNA, 14 bp, Artificial Sequence
  DNA oligonucleotide
ttttcgccgatggg

SEQ ID NO: 80 - DNA, 28 bp, Artificial Sequence
  DNA oligonucleotide
aacttcactgaagaagatggtgttgtag

SEQ ID NO: 81 - DNA, 50 bp, Artificial Sequence
  DNA oligonucleotide
ctgcttcctgcacgcacgttgtggacacgctcagggtgtgttggggtctt

SEQ ID NO: 82 - DNA, 36 bp, Artificial Sequence
  DNA oligonucleotide
tcctctcggcgcgtggcatcaacttcagcacgaggg

SEQ ID NO: 83 - DNA, 24 bp, Artificial Sequence
  DNA oligonucleotide
tttttgccgtaggggtcttgtttt

SEQ ID NO: 84 - DNA, 42 bp, Artificial Sequence
  DNA oligonucleotide
ggtacgtcgccttctgcttcaggatgttgccgtcctcctttt

SEQ ID NO: 85 - DNA, 41 bp, Artificial Sequence
  DNA oligonucleotide
tgtgatcgtgcagatggccctcgcggggtagcgaagtcgtg

SEQ ID NO: 86 - DNA, 44 bp, Artificial Sequence
  DNA oligonucleotide
tgggccagggcacgggatgtggtccctcgccggctgcgctcctg

SEQ ID NO: 87 - DNA, 25 bp, Artificial Sequence
  DNA oligonucleotide
cgggcagcagcacggggccgttttt

SEQ ID NO: 88 - DNA, 16 bp, Artificial Sequence
  DNA oligonucleotide
tttttagttgccgtcg

SEQ ID NO: 89 - DNA, 30 bp, Artificial Sequence
  DNA oligonucleotide
ctccttgccggtggcgcttctccgccctcg

SEQ ID NO: 90 - DNA, 42 bp, Artificial Sequence
  DNA oligonucleotide
tttttggcggacttgaaggctgaagcactgcacgccgttttt

SEQ ID NO: 91 - DNA, 21 bp, Artificial Sequence
  DNA oligonucleotide
ttttgaactccagcaggacca

SEQ ID NO: 92 - DNA, 24 bp, Artificial Sequence
  DNA oligonucleotide
gctcgatgttgtgcccgtcggcca

SEQ ID NO: 93 - DNA, 48 bp, Artificial Sequence
  DNA oligonucleotide
tgatatagtgccgtccggtagtggtcggcgagcagaggtagtggttgt

SEQ ID NO: 94 - DNA, 21 bp, Artificial Sequence
  DNA oligonucleotide
gacgtagccttcgggcatttt

SEQ ID NO: 95 - DNA, 13 bp, Artificial Sequence
  DNA oligonucleotide
ttttttgcgattt

SEQ ID NO: 96 - DNA, 29 bp, Artificial Sequence
  DNA oligonucleotide
ttttaggtcagggtggtggtcagcttttt

SEQ ID NO: 97 - DNA, 23 bp, Artificial Sequence
  DNA oligonucleotide
tggcggatcttgaagttcacttt

SEQ ID NO: 98 - DNA, 40 bp, Artificial Sequence
  DNA oligonucleotide
aaaaaccaccccgaacttgtggccgtttccgtaggtaaaa

SEQ ID NO: 99 - DNA, 40 bp, Artificial Sequence
  DNA oligonucleotide
aaaatggtcacgatgtggtcggggtagccttcgggcaaaa

SEQ ID NO: 100 - DNA, 40 bp, Artificial Sequence
  DNA oligonucleotide
aaaagccgtagatgaacttcacgatggtcctgggtcaaaa

SEQ ID NO: 101 - DNA, 31 bp, Artificial Sequence
  DNA oligonucleotide
aaaacttcacctcggcgcgggtcttttaaaa

SEQ ID NO: 102 - DNA, 26 bp, Artificial Sequence
  DNA oligonucleotide
aaaattttgtcggtagtgttttaaaa

SEQ ID NO: 103 - DNA, 38 bp, Artificial Sequence
  DNA oligonucleotide
aaaattttcgtgctgcttcagggtgggccattttaaaa

SEQ ID NO: 104 - DNA, 40 bp, Artificial Sequence
  DNA oligonucleotide
aaaatcttctgctgatatagacgttgtgttgtactcaaaa

SEQ ID NO: 105 - DNA, 38 bp, Artificial Sequence
  DNA oligonucleotide
aaaattttccggacacgctggtgaacagctttttaaaa

SEQ ID NO: 106 - DNA, 26 bp, Artificial Sequence
  DNA oligonucleotide
aaaattttaccttagttcttttaaaa

SEQ ID NO: 107 - DNA, 55 bp, Artificial Sequence
  DNA oligonucleotide
aaaaagcttgacgtcgccgtcccgatcgtcgcagctcgaccactttgctcaaaaa

SEQ ID NO: 108 - DNA, 40 bp, Artificial Sequence
  DNA oligonucleotide
aaaaatggcggagtcgtccttgaagaagccctcgaaaaaa

SEQ ID NO: 109 - DNA, 26 bp, Artificial Sequence
  DNA oligonucleotide
aaaatttttgtaggctgtttttaaaa

SEQ ID NO: 110 - DNA, 47 bp, Artificial Sequence
  DNA oligonucleotide
aaaattttcctcgcccttgctcaccatgttggggtggatgggcaaaa

SEQ ID NO: 111 - DNA, 58 bp, Artificial Sequence
  DNA oligonucleotide
aaaatcgatgcggttcaccagggtgtcgatggtgcgctcaggatgccccctggaaaaa

SEQ ID NO: 112 - DNA, 40 bp, Artificial Sequence
  DNA oligonucleotide
aaaaacgctgccttgtggcggatcttgagatgccgtaaaa

SEQ ID NO: 113 - DNA, 39 bp, Artificial Sequence
  DNA oligonucleotide
aaaagggcggacttgggtgctcaggtaggcagcagaaaa

SEQ ID NO: 114 - DNA, 40 bp, Artificial Sequence
  DNA oligonucleotide
aaaacgtagcggctgaagcacggccattgtctgcacaaaa

SEQ ID NO: 115 - DNA, 38 bp, Artificial Sequence
  DNA oligonucleotide
aaaattttttgtagttgcccttgaagaagtttttaaaa

SEQ ID NO: 116 - DNA, 26 bp, Artificial Sequence
  DNA oligonucleotide
aaaattttgatgcaagtcttttaaaa

SEQ ID NO: 117 - DNA, 37 bp, Artificial Sequence
  DNA oligonucleotide
ctcagtggacagccgttctggagcgttggacgaaact

(19) United States
(12) Patent Application Publication
(10) Pub. No.: US 2023/0392169 A1
(43) Pub. Date: Dec. 7, 2023

(54) NUCLEIC ACID NANOSTRUCTURES FOR DELIVERY OF NUCLEIC ACID SEQUENCES TO CELLS

(71) Applicant: UCL BUSINESS LTD, London (GB)

(72) Inventors: Stefan HOWORKA, London (GB); Jonathan BURNS, London (GB); Matthew REEVES, London (GB); Nishkantha ARULKUMARAN, London (GB); Mervyn SINGER, London (GB)

(21) Appl. No.: 18/246,447

(22) PCT Filed: Sep. 23, 2021
(86) PCT No.: PCT/GB21/52479

§ 371 Date: Mar. 23, 2023

(30) Foreign Application Priority Data
Sep. 23, 2020 (GB) 2015058.7

(51) Int. Cl. C12N 15/88; C12N 15/115

(52) U.S. Cl. CPC C12N 15/88; C12N 15/115; C12N 2310/16; C12N 2310/351

(57) ABSTRACT

Improved nucleic acid nanostructures provide a platform for stable and effective intra-cellular delivery of nucleic acids, suitably coding nucleic acids such as mRNA or ssDNA. A nucleic acid nanostructure is provided that comprises a first single stranded nucleic acid sequence that defines a scaffold sequence, wherein the scaffold sequence comprises at least one open reading frame that encodes a first gene product; and a plurality of single stranded nucleic acid sequences that define a plurality of staple sequences, wherein the plurality of staple sequences are capable of hybridising with one or more regions of the scaffold sequence in order to induce the formation of a geometrically defined higher order structure. The nanostructure may further comprise at least one membrane binding moiety, wherein the membrane binding moiety is configured to associate with a cell membrane. The nanostructures may be used in pharmaceutical compositions, such as vaccine compositions, and in methods of treating subjects in need thereof.

### FIELD OF THE INVENTION

The present invention relates to novel nucleic acid nanostructures and their uses. In particular, it relates to nanostructures for use in nucleic acid delivery to cells and tissues.

### BACKGROUND OF THE INVENTION

DNA nanostructures have shown potential to advance nanotechnology and the life sciences. Compared to other materials, DNA nanostructures have a highly controllable architecture which is based on predictable folding using base-pairing rules (Rothemund P. W. Nature 440, 297-302 (2006), Seeman, N. C.; Sleiman, H. F. Nat. Rev. Mater. (2017), 3, 17068; Hong, F. et al. Chem. Rev. 2017, 117, 12584-12640; Praetorius, F. et al. Nature (2017), 552, 84-87; Sacca, B.; Niemeyer, C. M. Angew. Chem. Int. Ed. 2012, 51, 58-66). By exploiting these properties, functional DNA nanostructures are increasingly designed to benefit areas outside DNA nanotechnology. Examples include DNA scaffolds which precisely position proteins and other biomolecular components for research applications in biophysics and molecular biology. Furthermore, predictable changes in DNA nanostructures have been exploited as smart biosensing devices which measure pH inside cells (Bhatia, D.; et al. Nat. Commun. (2011), 2, 339) or in cellular DNA nanocages for delivery of bioactive cargo (Walsh, A. S.; et al. ACS Nano (2011), 5, 5427-5432).

DNA nanostructures have been obtained for several designs including a structural core of six hexagonally arranged, interlinked DNA duplexes that enclose a hollow channel (see, for example, Douglas S. M., Marblestone A. H., Teerapittayanon S., Vazquez A., Church G. M., Shih W. M. Nucleic Acids Res. 37, 5001-5006 (2009); Zheng J., et al. Nature 461, 74-77 (2009); Fu J., et al. Nat. Nanotechnol. 9, 531-536 (2014); Burns J. R., et al. Angew. Chem. Int. Ed. 52, 12069-12072 (2013); Seifert A., Göpfrich K., Burns J. R., Fertig N., Keyser U. F., Howorka S. ACS Nano 9, 1117-1126 (2015); Langecker et al. (2012) Science, Vol. 338, Issue 6109, pp. 932-936; and Burns et al. (2013) Nano Lett., 13, 6, 2351-2356). Such nanostructures have been proposed for use as embedded membrane-spanning nanostructures whereby membrane insertion was achieved through equipping the structures' exterior with hydrophobic lipid anchors.

Circular nanotubes synthesized from DNA are also known in the art (Zheng et al. J. Am. Chem. Soc., 136, 10194-10197 (2014)).

Non-structured DNA and RNA have been developed into vaccines against cancer, with the SARS-CoV-2 pandemic having further increased the relevance of nucleic acids-based therapy platforms (MacKay et al. Nat. Commun. (2020), 11, 3523). A main advantage of mRNA type vaccines within a pandemic is the speed at which they can be developed and manufactured compared to traditional protein-based vaccines.

Delivery of naked nucleic acids, such as mRNA, is particularly challenging in the body as they are rapidly degraded by a range of extracellular enzymes such as DNases and RNases. Hence, to overcome these problems a range of strategies have been adopted in the art to improve in vivo and ex vivo delivery. Conventional approaches may include encapsulation of nucleic acids in drug delivery systems consisting of cationic molecules, lipids, polymers and/or biocompatible nanoparticles. These approaches may be further enhanced through use of physical transfection techniques such as electroporation or various ultrasonication methods. However, the use of complex encapsulation technologies and physical delivery strategies significantly increases cost. In addition, many encapsulation technologies are unstable at ambient temperatures necessitating onerous cold chain requirements, such as prolonged storage at −80° C. before administration. These requirements limit the applicability of the technology on the global scale, such as for vaccination in developing nations, and add considerably to wastage of valuable products.

RNA/DNA hybrid “origami” has been proposed for gene silencing therapy in which the RNA was folded into a nanostructure through RNA-DNA hybridization. After the incorporation of an active cell-targeting aptamer molecule, the tailored RNA/DNA hybrid origami demonstrated cellular uptake and controllable release of antisense RNAs in response to intracellular RNase H digestion within the cell (Wu et al. Nanoscale (2021) August 14; 13(30):12848-12853).

There is a need to further develop further improved and optimized methods and compositions for delivery of polynucleotide sequences, such as mRNA, in specific cells, organs and/or tissues. The present invention addresses the deficiencies in the art. These and other uses, features and advantages of the invention should be apparent to those skilled in the art from the teachings provided herein.

### SUMMARY OF THE INVENTION

In various embodiments, the invention provides compositions and methods suitable for delivering nucleotide-encoded products such as mRNA or ssDNA vector constructs, for example for use as vaccine and/or immunomodulatory compositions. The present invention provides improved nanostructures that provide a platform for stable and effective intra-cellular delivery of nucleic acids, suitably coding nucleic acids such as mRNA or ssDNA.

A first aspect of the invention provides a nucleic acid nanostructure comprising:
a first single stranded nucleic acid sequence that defines a scaffold sequence, wherein the scaffold sequence comprises at least one open reading frame that encodes a first gene product; and
a plurality of single stranded nucleic acid sequences that define a plurality of staple sequences, wherein the plurality of staple sequences are capable of hybridising with one or more regions of the scaffold sequence in order to induce the formation of a geometrically predefined higher order structure.

In a specific embodiment, the nanostructure further comprises at least one membrane binding moiety, wherein the membrane binding moiety is configured to associate with a cell membrane.

In a further embodiment, the nanostructure comprises at least a second single stranded nucleic acid sequence that defines a second scaffold sequence, and wherein the plurality of staple sequences are capable of hybridising with one or more regions of both the first and the second scaffold sequences in order to induce the formation of a geometrically predefined higher order structure. Optionally, the second scaffold sequence comprises at least one open reading frame that encodes a second gene product, wherein the second gene product may be the same or different to the first gene product.

In yet a further embodiment, the nanostructure comprises at least a third single stranded nucleic acid sequence that defines a third scaffold sequence, and wherein the plurality of staple sequences are capable of hybridising with one or more regions of the first, second and the third scaffold sequences in order to induce the formation of a geometrically predefined higher order structure. Optionally, the third scaffold sequence comprises at least one open reading frame that encodes a third gene product, wherein the third gene product may be the same or different to the first and/or second gene products.

In still further embodiments, the gene product is selected from one or more the group consisting of: an antigen; an immunomodulator; an antibody or a fragment thereof; an affimer (or other small affinity binding polypeptide); a cytokine; an enzyme; and a reporter protein. In a specific embodiment of the invention, the gene product comprises an ORF that encodes antigens that may include but are not limited to:
a SARS-CoV-2 spike protein, or a receptor binding domain (RBD) and any variant thereof
a human cytomegalovirus antigen—such as glycoprotein B, PP65 and/or IE1
a hepatitis C (HCV) antigen—such as E1 and E2 proteins
a human immunodeficiency virus (HIV) antigen—such as Gag and envelope proteins
a respiratory syncytial virus (RSV) antigen—such as F protein
an Ebola virus antigen—such as EBOV glycoprotein
a tuberculosis antigen—such as ESAT-6 and H37Rv proteins
a malaria antigen—such as circumsporozoite proteins and derivatives such as VMP001
a tumoral neoantigen—such as Wilms Tumour antigen (WT-1)
an influenza virus antigen

Suitably, the nucleic acid nanostructure of any one of the embodiments disclosed herein has a maximum dimension of less than around 100 nm, typically less than 50 nm, and suitably less than 20 nm.

A second aspect of the invention provides a RNA-DNA hybrid (RDH) nucleic acid nanostructure comprising
a first single stranded nucleic acid sequence that defines a scaffold sequence, wherein the scaffold sequence is comprised of RNA and includes at least one open reading frame that encodes a first gene product;
a plurality of single stranded DNA sequences that define a plurality of staple sequences, wherein the plurality of staple sequences are capable of hybridising with one or more regions of the scaffold sequence in order to induce the formation of a geometrically predefined higher order structure within the RDH; and
at least one hydrophobic membrane binding moiety, wherein the membrane binding moiety is configured to associate with a cell membrane.

Suitably, the RNA is a messenger RNA (mRNA).

A third aspect of the invention provides a pharmaceutical composition comprising a nanostructure as described herein in combination with a suitable excipient.

A fourth aspect provides the pharmaceutical composition for use as a vaccine. In one embodiment the nanostructure comprises an open reading frame that encodes a gene product that is an antigen. Optionally, the antigen is derived from an infectious pathogen selected from the group consisting of: a virus; a bacterium; a fungus; a protozoan; a prion; and a helminth. Suitably, the antigen comprises all or part of any variant of the spike protein of the SARS-CoV-2, typically the antigen comprises all or a part of a spike protein Receptor Binding Domain (RBD). Thus, the composition may be for prophylactic use in the prevention of COVID-19 disease. Alternatively, the antigen is derived from a tumour. such that, in one embodiment, the pharmaceutical composition is for use in treating cancer.

A fifth aspect provides a method of treating a subject in need thereof, comprising administering to the subject, suitably a human subject, a pharmaceutical composition as described herein.

A sixth aspect of the invention provides for an ex vivo method of treating a subject in need thereof, comprising administering to a sample of cells obtained from the subject a nanostructure as described herein. Typically, the cells are reintroduced to the subject following exposure to the nanostructure. Optionally, the cells comprise immune cells, suitably the cells comprise white blood cells (WBCs).

A seventh aspect of the invention provides a biostable delivery vector for initiating polypeptide translation within an animal cell, the vector comprising:
(a) a mRNA sequence that comprises at least one ORF in operative combination with at least one flanking untranslated region (UTR), a 5′ cap and a poly-adenosine tail, wherein the ORF encodes a polypeptide; and
(b) a plurality of DNA oligonucleotide sequences, wherein the plurality of DNA oligonucleotide sequences comprise sequences that are complimentary to regions of the mRNA sequence;
wherein the plurality of DNA oligonucleotide sequences hybridise to the mRNA and thereby induce formation of a defined geometric secondary structure that imparts resistance to nuclease digestion.

An eighth aspect of the invention provides a method of manufacturing a biostable delivery vector for initiating polypeptide translation within an animal cell, the method comprising:
providing a mRNA sequence that comprises at least one ORF in operative combination with at least one flanking untranslated region (UTR), a 5′ cap and a poly-adenosine tail, wherein the ORF encodes a polypeptide, a plurality of DNA oligonucleotide sequences, wherein the plurality of DNA oligonucleotide sequences comprise sequences that are complimentary to regions of the mRNA sequence; and
combining the mRNA sequence with the plurality of DNA oligonucleotide sequences under conditions that facilitate hybridisation and RNA-DNA duplex formation, thereby creating an RDH nanostructure.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### DESCRIPTION OF THE DRAWINGS

One or more embodiments of the invention will now be described, by way of example only, with reference to the accompanying drawings, in which:

FIG. 1 is a representation of an embodiment of the invention in which nucleic acid nanobarrel structures containing cholesterol lipid anchors show selective binding and immunosuppression properties with white blood cells (WBCs) composed of peripheral blood mononuclear cells (PBMCs) and granulocytes, rather than red blood cells (RBCs).

FIG. 2 is a representation of an embodiment of the invention that shows cholesterol-tagged DNA nanobarrels selectively interact with white blood cells in the multi-cell environment from human blood. (A) DNA nanobarrels containing cholesterol lipid anchors selectively interact with white blood cells (WBCs) composed of peripheral blood mononuclear cells (PBMCs) and granulocytes, rather than red blood cells (RBCs). (B) Top and side view of DNA nanostructures used in one embodiment and their expected membrane interaction, including, from left to right, control barrels NB-0C and NB-1C, and active barrels NB-3C, which are expected to not interact, tether, and span lipid bilayers, respectively. Additional control structures encompass NB-3C-1, which was expected to tether but not span lipid bilayers, and single duplexes without and with a single cholesterol anchor.

FIG. 3 shows results that demonstrate DNA nanobarrels' highly selective binding to immune cells according to one embodiment of the invention. (A) Confocal laser scanning microscopy (CLSM) analysis of DNA nanobarrels (magenta channel) (500 nM) with WBC (blue channel) in Hank's buffered saline (HBSS), scale bar 10 μm. (B-I) CLSM time series of WBCs upon addition of NB-3C (500 nM) in HBSS, NB-3C was added between the first and second frames, where each frame represents a 5-minute interval and measures 12 μm×12 μm. (B-II) CLSM-derived membrane binding kinetics of NB-0C (bottom line), NB-1C (middle line) and NB-3C (top line) to WBCs. (C) Pre-treatment of WBCs with cytochalasin-D (CD), an inhibitor of actin cytoskeleton and phagocytosis, did not result in a significant reduction of DNA nanobarrels associated with polymorphonuclear cells. (D) Flow cytometric analysis comparing different barrel constructs' binding (250 nM) and localization towards (D-I) granulocytes and (D-II) PBMCs. (D-III) In HBSS significantly greater binding to granulocytes compared to PBMCs was observed for NB-3C and NB-3C-1 constructs compared to NB-0C. Data represents median and error bars represent interquartile range of experiments conducted with 6-10 biological replicates; apart from c, which was done with 3 technical replicates (*p<0.05).

FIG. 4 shows results that demonstrate that DNA nanobarrels do not porate white blood cells according to one embodiment of the invention. (A) Schematic representation of membrane-spanning alignment of NB-3C. (B) CLSM demonstrated strong NB-3C (magenta channel) binding to GUVs, scale bar 10 μm. (C) Flow cytometry analysis of barrel constructs identifying Atto-647 influx towards giant unilamellar vesicles (GUVs). (D) Schematic representation of NB-3C in a membrane-tethered orientation. (E) Fluorescence activated cell sorting analysis of barrel constructs identifying Atto-647 flux towards (E-I) granulocytes and (E-II) PBMCs demonstrating no significant dye uptake. Data represents median and error bars represent interquartile range of experiments conducted with 3 technical replicates; apart from (C), which was done with 6-9 biological replicates, each with 3 technical replicates (*p<0.05). The expected binding of NB-3C to GUVs and associated dye transport was confirmed by CLSM (FIG. 4B) and FACS (FIG. 4C), respectively. The membrane activity of NB-3C was due to the cholesterol anchors as the negative control NB-0C did not induce flow (FIG. 4C).

FIG. 5 shows results that demonstrate DNA nanobarrels do not affect the viability of white and red blood cells according to one embodiment of the invention. (A, B) Viability assays in serum-free conditions after 6 h for (A) granulocytes and (B) PBMCs demonstrate no significant loss in cell viability compared to untreated controls. (C-I) CLSM images of NB-3C with RBCs in HBSS, scale bar 10 μm. (C-II) UV-vis absorbance spectra of RBCs with either NB-3C (purple), NB-1C (pink) and NB-OC (grey), and controls PBS (orange) and lysed RBCs (black) in HBSS. Data represents median and error bars represent interquartile range of experiments conducted with three biological replicates (*p<0.05).

FIG. 6 shows results of the immune response of DNA nanobarrels ex vivo according to one embodiment of the invention. (A) Flow cytometry analysis showing reactive oxygen species (ROS) production in serum-free conditions for (A-I) granulocytes (p=0.41) and (A-II) PBMCs (p=0.056) following 90 min incubation. (B-I) In whole blood, release of cytokines occurs after 90 min and peaks at 6 h; (B-II) a dose-dependent increase in pro-inflammatory cytokine TNF-alpha is seen following 6 h of incubation with NB-3C in whole blood. (C) Incubation of immune cells with DNA nanobarrels for 90 min followed by addition of lipopolysaccharide (LPS) for 5 h results in a cholesterol-dependent reduction in LPS-induced pro-inflammatory cytokine release (C-I) IL-6 and (C-II) TNF-alpha. Data represents median and error bars represent interquartile range of experiments conducted with 3-9 biological replicates (or 2 biological replicates for b); each with 3 technical replicates (*p<0.05).

FIG. 7 show 2D maps of DNA nanobarrels according to one embodiment of the invention. (A) NB-0C, (B) NB-1C, (C) NB-3C, and (D) NB-3C-1. The component DNA strands are represented as lines, where the 5′ and 3′ termini are indicated by squares and triangles, respectively. The segments in dashed lines at the top and bottom of the 2D maps indicate T4 single-strand loops. The solid circles show the positions for the cholesterol modifications. The hexagon and star denote the position of TAMRA and FAM fluorophores, respectively in (A).

FIG. 8 shows a photograph of 10% SDS PAGE analysis of DNA nanobarrel formation according to one embodiment of the invention in PBS, where M denotes the 100 bp size marker lane.

FIG. 9 shows graphs of results that demonstrate the thermal stability of DNA nanobarrels according to embodiments of the present invention. (a) Fluorescence emission spectra of FAM (donor) and TAMRA (acceptor), and FAM only or TAMRA only labelled NB-3C, excitation at 495 nm. (b) Fluorescence donor emission melting profile of FAM and TAMRA labelled NB-OC in human serum. (c-i) NB-OC UV absorbance melting profile with either 0, 0.5 or 5 U per mL of DNAse I, incubated for 4 h at 37° C. in DNAse I buffer, (c-ii) corresponding differential profiles.

FIG. 10 shows photographs of 2% Agarose gel electrophoretic analysis of DNA nanostructures NB-0C, NB-1C, and NB-3C according to embodiments of the invention that were incubated in (a) PBS, (b) human serum and (c) whole blood for 4 h at 37° C. The maximum extent possible digestion was determined with DNase I digestion (400 U per mL) and is shown in lanes labelled with D. M denotes the 100 bp marker.

FIG. 11 shows photographs of 1.5% Agarose gel analysis NB-OC according to embodiments of the invention incubated with different concentrations of DNAse I per mL for 20 min (top row) or 4 h at 37° C. M denotes the 100 bp marker.

FIG. 12 shows CLSM analysis of nanostructure embodiment NB-3C binding to immune cells in different media. (a) NB-3C (magenta channel) with WBCs in either Hank's buffered saline (HBSS), human serum (HS) or heat-inactivated HS, binding is only observed in HBSS. (b-i) NB-3C incubated with WBCs in HBSS (left) then transferred into HS (right), and (b-ii) relative membrane fluorescence of each condition taken from b-i. All scale bars, 25 μm. Data represents median and interquartile range of the cell membrane fluorescence, n=6.

FIG. 13 shows CLSM analysis of WBCs (blue channel) incubated for 90 min with nanostructure embodiment NB-3C (gray channel) in HBSS. The majority of the NB-3C DNA nanobarrels remain localized to the cell membrane (arrowhead) though some DNA nanobarrels are internalized and co-localized with lysosomes (green channel) (arrow) as shown in the merge zoom panel. All scale bars, 10 μm.

FIG. 14 shows the results of flow cytometry-derived binding kinetics of DNA nanobarrels according to embodiments of the invention to immune cells in whole blood (a) Characteristic forward and side scatter profiles used to gate granulocyte and PBMC populations. (b) Flow cytometry profiles of Alexa647-conjugated DNA nanobarrels. (c) Flow cytometry analysis comparing binding and localization of different DNA constructs in whole blood to either (c-i) granulocytes, (c-ii) PBMC, and (c-iii) comparing the ratios of granulocytes vs PBMCs binding for each DNA construct. Data represents median and interquartile range, and the experiments were conducted with >6 biological replicates.

FIG. 15 shows graphs that present the results of experiments that identify the effect of DNA nanobarrels according to embodiments of the invention on immune cells ex vivo. (a) Flow cytometry analysis quantifies the production of reactive oxygen species (ROS) in (a-i) granulocytes and (a-ii) PBMCs in whole blood following a 90 min incubation with DNA nanostructures. (b-i) NB-0C induces a strong release pro-inflammatory cytokine following 6 h incubation. (b-ii). Immune cells in whole blood (+ Serum) have significantly greater TNF-alpha release in response to LPS compared to immune cells re-suspended in HBSS (− Serum). (c) Incubation of immune cells with DNA nanobarrels prior to PMA stimulation results in a cholesterol-independent lowered ROS production. Data represents median and interquartile range, and the experiments were conducted with >3 biological replicates.

FIG. 16 shows a graph that presents the results of a whole blood stimulation revealing that the reduction in TNF-alpha release is greater with nanobarrel embodiment NB-3C compared to cholesterol-PEG. Immune cells were incubated with cholesterol-PEG 600 at 750 nM or NB-3C at 250 nM for 90 min followed by addition of 100 ng/mL lipopolysaccharide (LPS) for 5 h. Data represents median and interquartile range, and the experiments were conducted with >3 biological replicates.

FIG. 17 shows schematic diagrams of Mfold predicted structure of (A) GFP mRNA, and 2D maps of GFP mRNA-DNA origami nanostructures of embodiments of the invention assayed, where the 5′ and 3′ terminus are represented as a square and triangle, respectively (B) shows linear duplex conformation; (C) shows a helical bundle conformation; (D) a square plate conformation; and (E) shows a cube conformation.

FIG. 18 shows a photograph of a 1.5% agarose gel electrophoretic analysis of GFP mRNA and GFP mRNA-DNA origami nanostructures, as depicted in FIG. 17, with 1:5 and 1:1 scaffold to staple ratios, respectively. The 1K bp M and 100 bp M represent the 1000 base pair and 100 base pair DNA markers lanes, respectively.

FIG. 19 is a graph that shows the results of size exclusion chromatography analysis of GFP mRNA (dashed line), GFP mRNA-DNA square plate (SP) (solid line) and square plate staples (dotted line).

FIG. 20 shows (A) an atomic force microscope image of SEC purified SP, scale bar 50 nm, and (B) a corresponding graph that shows cross-section analysis of dotted lines.

FIG. 21 shows a photograph of a gel electrophoretic analysis on the stability of the mRNA-DNA square plate (SP) under conditions (A) 0, 3 or 6× freeze-thaw cycles, (B) nuclease digestion with the stated amount of RNase H, and (C) in the stated amount of CaCl₂). Annotated 100 bp marker bands are shown on the left.

FIG. 22 is a graph showing cell-free in vitro expression of GFP from the GFP mRNA nanostructures of FIG. 17 after 24 hrs at 37° C. mRNA-DNA linear duplex (LD), mRNA-DNA cube (CB), mRNA-DNA square plate (SP) and mRNA-DNA helical bundle (HB).

FIG. 23 is a graph showing the results of a nuclease stability assay of GFP mRNA and GFP mRNA-DNA square plate (SP). The samples were incubated with or without RNase for 30 mins at 37° C., followed by addition of cell-free in vitro expression reagents for the production of GFP.

FIG. 24 shows Fluorescent microscopy images of HeLa cells 48 hours post transfection showing GFP fluorescence for GFP mRNA, HSP, HSP-18 and HSP-22 (200× magnification).

FIG. 25 shows graphs that display the results of human HeLa cells transfected with DNA:RNA nano-structures of embodiments of the invention that express GFP. At 48 hours post transfection, cells were analysed by flow cytometry for GFP expression and the number of GFP positive cells (A) or Mean fluorescent Intensity (MFI) of GFP expression (B) was calculated. Human HeLa cells were transfected with GFP mRNA or constructs HSP, HSP-18, HSP-22, or non-binding SS. The ratio of GFP mRNA to DNA staples was either 1:5 (left of panels) or 1:1 (right half of panels). As control, cells transfected with GFP mRNA alone previously demonstrated to express GFP (pos control). Background GFP expression levels was from cells not transfected with GFP mRNA (lipo control).

### DETAILED DESCRIPTION OF THE INVENTION

Prior to setting forth the invention, a number of definitions are provided that will assist in the understanding of the invention.

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, NY; Ausubel, F. M. et al. (Current Protocols in Molecular Biology, John Wiley & Sons, Online ISSN:1934-3647); B. Roe, J. Crabtree, and A. Kahn, 1996, DNA Isolation and Sequencing: Essential Techniques, John Wiley & Sons; J. M. Polak and James O′D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press; Synthetic Biology, Part A, Methods in Enzymology, Edited by Chris Voigt, Volume 497, pages 2-662 (2011); Synthetic Biology, Part B, Computer Aided Design and DNA Assembly, Methods in Enzymology, Edited by Christopher Voigt, Volume 498, Pages 2-500 (2011); RNA Interference, Methods in Enzymology, David R. Engelke, and John J. Rossi, Volume 392, Pages 1-454 (2005). Each of these general texts is herein incorporated by reference.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term ‘modular’ as used herein refers to the use of one or more units, or modules, to design or construct a whole or part of a larger system. In the context of the present invention it refers to the use of individual modules, sub-units or building blocks to construct a nanostructure, suitably a nanostructure intended to effect delivery of a coding or non-coding nucleic acid to a cell. The modules may be each the same or the modules may be different. To form the nanostructure, the individual modules may be connected or inter-linked to one or more other modules. The means of connection between modules may be by chemical or physical means, such as covalent or non-covalent chemical bonding or by electrostatic or other attractive forces. Alternatively, or in addition, the means of connection may be via an additional module, bracing member, portion or linkage. The modular design of a nanostructure may comprise a frame or framework of modules, and additional, typically smaller, sub-modules that connect, or support the frame, acting as struts or bracing members. The modules or sub-modules may be formed of nucleic acids, typically DNA, RNA and synthetic nucleic acids or analogues thereof (e.g. LNA or PNA). Each individual unit may be assembled by DNA/RNA origami techniques described elsewhere herein using suitably selected scaffold and staple strands in order to create a higher order structure—e.g., a secondary structure having defined geometric parameters. In specific embodiments of the invention, the nanostructure is comprised of modules that integrate the nucleic acid that is to be delivered—i.e. the cargo—into the structure itself.

The term ‘nucleic acid’ as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may include DNA and RNA, and are typically manufactured synthetically, but may also be isolated from natural sources. Nucleic acids may further include modified DNA or RNA, for example DNA or RNA that has been methylated or that has been subject to chemical modification, for example 5′-capping with 7-methylguanosine or analogues thereof, 3′-processing such as cleavage and polyadenylation, and splicing, or labelling with fluorophores or other compounds. Nucleic acids may also include synthetic nucleic acids (XNA) or nucleic acid analogues, such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Hence, where the terms DNA′ and ‘RNA’ are used herein it should be understood that these terms are not limited to only include naturally occurring nucleotides. Sizes of nucleic acids, also referred to herein as ‘polynucleotides’ are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 100 nucleotides in length are typically called ‘oligonucleotides’.

As used herein, the terms ‘3″ (‘3 prime’) and ‘5″ (‘5 prime’) take their usual meanings in the art, i.e. to distinguish the ends of polynucleotides. A polynucleotide has a 5′ and a 3′ end and polynucleotide sequences are conventionally written in a 5′ to 3′ direction. The term ‘complements of a polynucleotide molecule’ denotes a polynucleotide molecule having a complementary base sequence and reverse orientation as compared to a reference sequence.

The term ‘duplex’ is used herein refers to double-stranded DNA (dsDNA), meaning that the nucleotides of two complimentary DNA sequences have bonded together and then coiled to form a double helix, and also single-stranded RNA (ssRNA) that has annealed to a complimentary DNA sequence to generate an RNA-DNA hybrid (RDH) duplex. An RDH nanostructure may comprise a single RNA scaffold sequence with multiple shorter hybridised DNA sequences (e.g. DNA oligonucleotides) acting as staples forming a series of RDH duplexes along the length of the RNA scaffold thereby defining higher order structures.

According to the present invention, homology to the nucleic acid sequences described herein is not limited simply to 100%, 99%, 98%, 97%, 95% or even 90% sequence identity. Many nucleic acid sequences can demonstrate biochemical equivalence to each other despite having apparently low sequence identity. In the present invention homologous nucleic acid sequences are considered to be those that will hybridise to each other under conditions of low stringency (Sambrook J. et al, Molecular Cloning: a Laboratory Manual, Cold Spring Harbor Press, Cold Spring Harbor, NY). However, it may be desired in some cases to distinguish between two sequences which can hybridise to each other but contain some mismatches—an “inexact match”, “imperfect match”, or “inexact complementarity”—and two sequences which can hybridise to each other with no mismatches—an “exact match”, “perfect match”, or “exact complementarity”. Further, possible degrees of mismatch are considered.

As used herein, the term ‘nanostructure’ refers to a geometrically predefined or ‘predesigned’ two or three dimensional molecular structure typically comprised from a biopolymer, suitably a naturally or non-naturally occurring nucleic acid, which structure has at least one dimension or an aspect of its geometry that is within the nanoscale (i.e. 10-9 metres). Nanoscale structures suitably have dimensions or geometry of less than around 100 nm, typically less than around 50 nm, and most suitably around 20 nm. Nanoscale structures suitably possess dimensions or geometry greater than around 0.1 nm, typically greater than around 1 nm, and optionally greater than around 2 nm. Assembly of nucleic acid nanostructures may occur spontaneously in solution, such as by heating and cooling a mixture of DNA strands of preselected sequences, or may require presence of additional co-factors including, but not limited to, nucleic acid scaffolds, nucleic acid aptamers, nucleic acid staples, co-enzymes, and molecular chaperones. Where desired nanostructures result from one or more predesigned spontaneously self-folding nucleic acid molecules, such as DNA or RNA, this is typically referred to as nucleic acid ‘origami’. Rational design and folding of DNA to create two dimensional or three dimensional nanoscale structures and shapes is known in the art (e.g. Rothemund (2006) Nature 440, 297-302). The term ‘geometrically predefined’ is used to mean that the geometry of the nanostructure is predefined such that upon assembly the nanostructure conforms to the desired shape and configuration intended by the designer. By way of example, selection of the scaffold and staple sequences is such that the rational design of the nanostructure is assured repeatedly upon completion of hybridisation. In embodiments of the invention the nanostructure is geometrically predefined to form a planar structure such as a circle, triangle, square, rectangle or other regular or irregular polygon; or a three dimensional structure such as a spheroid, a barrel, a cone, a pyramid, a cuboid or other regular or irregular polyhedron.

In the classical scaffold-and-staple approach, one or more long biogenic scaffold strand component(s) is folded into a defined nucleic acid nanostructure with a staple component consisting of shorter synthetic staple oligonucleotides. Classical DNA nanostructures are formed of bundles of parallel aligned DNA duplexes that are arranged into polygons that enclose a channel and puncture a membrane bilayer. Suitably certain scaffold structures may be based off M13 or phiX174 sequences, which a plurality of smaller staple and linker sequences configured to achieve the desired three-dimensional nanostructural geometry. In embodiments of the present invention alternative scaffolds may be utilised, particularly where the scaffold sequence also serves the function of a nucleic acid that is to be delivered to a cell, such as a gene therapy vector, viral genome, or mRNA sequence. In these embodiments, the scaffold comprises at least one gene expression cassette, that will suitably include at least one ORF. The at least one ORF codes for a gene product, such as a polypeptide or protein, that maybe translated and assembled within a target recipient cell.

The nucleic acid sequences that form the nanostructures will typically be manufactured synthetically, although they may also be obtained by conventional recombinant nucleic acid techniques. DNA constructs comprising the required sequences may be comprised within vectors grown within a microbial host organism (such as E. coli). This would allow for large quantities of DNA or RNA to be prepared within a bioreactor and then harvested using conventional techniques. The vectors may be isolated, purified to remove extraneous material, with the desired DNA sequences excised by restriction endonucleases and isolated, such as by using chromatographic or electrophoretic separation. Hence, according to embodiments of the invention a method of manufacturing a biostable nanostructure, that may function as an delivery vector, is provided for initiating polypeptide translation within an animal cell. In this embodiment, one or more mRNA sequences are combined with a plurality of DNA oligonucleotide sequences under conditions that facilitate hybridisation and RNA-DNA duplex formation, thereby creating an RDH nanostructure having improved biostability.

A ‘polypeptide’ is a polymer of amino acid residues joined by peptide bonds, whether produced naturally or in vitro by synthetic means. Polypeptides of less than around 12 amino acid residues in length are typically referred to as ‘peptides’ and those between about 12 and about amino acid residues in length may be referred to as ‘oligopeptides’. The term ‘polypeptide’ as used herein denotes the product of a naturally occurring or recombinantly modified polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term ‘protein’ is used herein to refer to a macromolecule comprising one or more polypeptide chains. In embodiments of the present invention, two or more polypeptides may be delivered to a recipient cell that encode subunits or domains of a larger protein, which assemble within the cell to form the ultimate protein gene product.

As used herein the term ‘hydrophobic’ refers to a molecule having apolar character including organic molecules and polymers. Examples are saturated or unsaturated hydrocarbons. The molecule may have amphipathic properties.

As used herein, the term ‘hydrophobically-modified’ relates to the modification (joining, bonding or otherwise linking) of a polynucleotide strand with one or more hydrophobic moieties. A ‘hydrophobic moiety’ as defined herein is a hydrophobic organic molecule. The hydrophobic moiety may be any moiety comprising non-polar or low polarity aliphatic, aliphatic-aromatic or aromatic chains. Suitably, the hydrophobic moieties utilised in the present invention encompass molecules such as long chain carbocyclic molecules, polymers, block co-polymers, and lipids.

The term ‘lipids’ as defined herein relates to fatty acids and their derivatives (including tri-, di-, monoglycerides, and phospholipids), as well as sterol-containing metabolites such as cholesterol. The hydrophobic moieties comprised within the embodiments of the present invention are capable of forming non-covalent attractive interactions with phospholipid bilayers, such as the lipid-based membranes of cells and act as membrane anchors for the nanostructure. According to certain embodiments of the present invention suitable hydrophobic moieties, such as lipid molecules, possessing membrane anchoring properties may include sterols (including cholesterol, derivatives of cholesterol, phytosterol, ergosterol and bile acid), alkylated phenols (including methylated phenols and tocopherols), flavones (including flavanone containing compounds such as 6-hydroxyflavone), saturated and unsaturated fatty acids (including derivatives such as lauric, oleic, linoleic and palmitic acids), and synthetic lipid molecules (including dodecyl-beta-D-glucoside). The anchors for the polymer membrane may be the same as for lipid bilayers or they may be different. The specific hydrophobic moiety anchor may be selected based on the binding performance of the membrane chosen.

In embodiments of the invention the disclosed nanostructures may comprise one or more hydrophobic or lipophilic anchors that act to attach or connect or anchor the hydrophilic nucleic acid nanostructure to a generally hydrophobic membrane such as the lipid bilayer of a cell. The lipid anchors are attached to the nanostructure or comprised within modules that form part of overall the nanostructure. Suitably attachment is via oligonucleotides that carry the lipid anchor, suitably cholesterol, at the 5′ or 3′ terminus. Polynucleotides or oligonucleotides may be functionalized using a modified phosphoramidite in the strand synthesis reaction, which is easily compatible for the addition of reactive groups, such as cholesterol and lipids, or attachment groups including thiol and biotin. Enzymic modification using a terminal transferase can also be used to incorporate an oligonucleotide, which incorporates a modification such as an anchor, to the 3′ of a single stranded nucleic acid (e.g. ssDNA). These lipid modified anchor strands may hybridize via ‘adaptor’ oligonucleotides to corresponding sections of the nucleic acid sequence forming the scaffold section of the nanostructure. Alternatively, the lipid anchors are assembled with the nanostructure using lipid-modified oligonucleotides that contribute as either the scaffold or staple strands. A combination of approaches to anchoring using two or more membrane anchors may also be adopted wherein anchors are incorporated into one or all of a scaffold strand, a staple strand and an adaptor oligonucleotide. Cholesterol has been found to be a particularly suitable lipid for use as an anchor in the present invention. The use of other lipids as anchors is contemplated, although it may be expected that there is a particular preference for a particular lipid, and a given number of membrane anchors, for a given cell membrane.

In an alternative embodiment of the invention, the hydrophobic modification is comprised within one or more synthetic nucleic acids (XNAs) incorporated into the nanostructure structure itself.

The nanostructures of the present invention will comprise a nucleic acid sequence which is intended to be delivered to a cell or tissue that is comprised of cells. Typically the nucleic acid sequence intended for delivery is termed a ‘cargo’ sequence which may also be synonymous with the terms ‘delivery sequence’ or ‘coding sequence’ as appropriate. According to a specific embodiment of the present invention the cargo sequence comprises an expression construct. An expression construct may comprise a plurality of sequences that are arranged sequentially so that they function cooperatively in order to achieve their intended purposes. By way of example, a cargo that comprises a DNA vector will typically include a promoter sequence that allows for initiation of transcription that proceeds through a linked coding sequence as far as a termination sequence.

In the case of a cargo that comprises an RNA sequence, the RNA may be a linear or circular mRNA. The RNA may comprise one or more untranslated regions (UTRs) may be arranged in relation to a linked polypeptide coding sequence i.e. the open reading frame (ORF). A given mRNA as disclosed herein may comprise more than one ORFs, a so-called polycistronic RNA. An mRNA may encode more than one polypeptide, and may as a result include cleavage sites or other sequences necessary to result in the production of multiple functional products, as known in the art. A UTR may be located 5′ or 3′ in relation to an operatively linked coding sequence ORF. UTRs may comprise sequences typically found in mRNA sequences found in nature, such as any one or more of: Kozak consensus sequences, initiation codons, cis-acting translational regulatory elements, cap-independent translation initiator sequences, poly-A tails, internal ribosome entry sites (IRES) such as those derived from foot and mouth disease virus or poliovirus, structures regulating mRNA stability and/or longevity (such as miRNA binding sequences), sequences directing the localisation of the mRNA, and so on. An mRNA may comprise multiple UTRs that are the same or different. UTRs may comprise linear sequences that provide intra-cellular translational or stability control over the mRNA, such as Kozak sequences, or they may also comprise one or more sequences that promote the formation of localised secondary structure, particularly within a 5′ UTR. In one embodiment of the invention, a 5′ UTR that has a lower-than-average GC content may be utilised to promote efficient translation of the mRNA once processed within the recipient cell. Hence, embodiments of the invention provide a biostable delivery vector for initiating polypeptide translation within a recipient cell whereby formation of a defined geometric nanostructure imparts resistance to nuclease digestion as well as improved thermal stability.

The term ‘expressing a polypeptide’ in the context of the present invention refers to the biosynthetic production of a polypeptide for which the cargo polynucleotide sequences, either as DNA or RNA, described herein code. Typically, this involves translation of a cargo mRNA sequence—i.e. the ORF—by the ribosomal machinery of the recipient cell to which the sequence is delivered. Hence, the expressed polypeptide may represent a gene product encoded by the mRNA such as a peptide, polypeptide or protein. Where a particular protein consists of more than one subunit, the mRNA may code for one or more than one subunit within one or more ORFs. In alternative embodiments, one or more nanostructures may comprise at least a first mRNA that codes for a first subunit, whilst a second co-administered mRNA either within the same or a different nanostructure may code for a second subunit that, when translated in situ, leads to assembly of a multi-subunit protein gene product within the recipient cell. Translation of the gene product within the target cell allows for localised post-translational modification appropriate to the cell type to be applied. Such modifications may regulate folding, localization, interactions, degradation, and activity of the gene product. Typical post translational modifications may include cleavage, refolding and/or chemical modification such as methylation, acetylation, sumoylation or glycosylation.

In specific embodiments of the invention, a nanostructure may comprise one, two, three, four or more mRNA sequences all or some of which may comprise an ORF that encodes a gene product. In this way the nanostructure may serve to deliver a plurality of cargo mRNA sequences to a recipient target cell.

Delivery of cargo mRNA directly to cells allows direct and controllable translation of the desired gene products such as polypeptides and/or proteins in the cells. Provision of mRNA specifically allows not only for the use of cell expression modulation mechanisms but also represents a finite and exhaustible supply of the product, rather than the potentially permanent change to the transcriptome of a target cell, which an episomal or genomically inserted DNA vector might initiate.

In embodiments of the present invention the cargo nucleic acid that is comprised within a specified nanostructure may function as an mRNA that codes for gene product having or that contributes towards a therapeutic or diagnostic effect within an individual recipient—e.g. a human or animal subject. The therapeutic or diagnostic effect may be caused by the gene product itself, or by another component of the therapeutic intervention that cooperates or interacts with the gene product in vivo. In one embodiment, the subject to whom therapy is administered is a mammal (e.g., rodent, primate, non-human mammal, domestic animal or livestock, such as a dog, cat, rabbit, guinea pig, cow, horse, sheep, goat and the like), and is suitably a human. In embodiments of the invention the subject may be indicated as a recipient for a therapeutic treatment, such as vaccination, therapeutic viral therapy, chemotherapy, radiation therapy, targeted therapy, adoptive cell therapy and/or anti-immune checkpoint therapy. The terms “treating”, “therapy” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

In other embodiments of the invention, the nanostructures may be delivered to animal or non-animal cells, such as plant cells, fungi, bacteria and archaea. In such embodiments, the nanostructures may serve as a delivery vector for a coding nucleic acid cargo that is to be transfected into the recipient cells. Animal cells may include mammalian, avian, reptilian, fish, insect or amphibian cells. Examples for plant species of interest are monocotyledonous plants like wheat, maize, rice, barley, oats, millet and such like as well as dicotyledonous plants like rape seed, canola, sugar beet, soybean, peas, alfalfa, cotton, sunflower, potato, tomato, and tobacco. Microbial plant species may include algal species of the genus Nannochloropsis, Chlamydomonas, Scenedesmus, or Dunaliella. Other microbial cells may include yeast of the genus Saccharomyces or Pichia.

In a specific embodiment of the invention the nanostructures are suitable for delivery of a cargo nucleic acid sequence to cells that are part of the immune system (which can be either the adaptive or the innate immune system) of a recipient subject. Examples of immune cells include, but are not limited to T cells, B cells, granulocytes, NK cells, NKT cells, lymphocytes, dendritic cells, myeloid cells, as well as suitable progenitor cells, including stem cells and/or iPSCs. Particularly envisaged immune cells include white blood cells (leukocytes), including lymphocytes, monocytes, macrophages, granulocytes, B cells and dendritic cells. Delivery may occur in vivo, such as by direct injection into a subject, or ex vivo such as following leukophoretic harvesting of stem cells from a subject.

A nanostructure according to an embodiment of the present invention is able to associate with, or bind to, a cell a membrane or to a microsomal or exosomal structure within the body of a subject. In an embodiment of the invention, the nanostructure associates with a membrane via insertion of a least one associated hydrophobic anchor moiety into the membrane bilayer. According to this embodiment of the invention a majority of the nanostructure is localised to an outer surface of the membrane but does not penetrate or puncture the membrane in the manner of a membrane-spanning nanopore.

Suitably the nanostructures of the invention comprise one or more polynucleotide strands that provide a functional scaffold component, wherein the polynucleotide strands comprised within the scaffold component include a polynucleotide backbone; and a plurality of polynucleotide strands that provide a plurality of functional staple components. The scaffold strand(s) cooperate with and hybridise to the plurality of staple polynucleotide strands—e.g. via appropriate Watson-Crick base pairing hybridisation—in order to form a three-dimensional configuration of the nanostructure.

A nanostructure according to an embodiment of the invention may comprise a nucleic acid nanostructure such as a nanobarrel or nanoraft, which is typically a rectangular, regular or irregular polygonal, circular, cuboid or ellipsoid substantially planar nanostructure. Hence, the nucleic acid duplexes are formed into a bundle, or a series of modules comprised of bundles of duplexes, that cooperate to define the desired geometry of nanostructure.

The nanostructures of all configurations of the present invention may be assembled via the ‘scaffold-and-staple’ approach. In this important route to nucleic acid nanostructures, DNA or RNA is utilized as a building material in order to make nanoscale three dimensional shapes. Assembly of these complex nanostructures from a plurality of un-hybridized linear molecules is typically referred to as ‘nucleic acid origami’. The nucleic acid origami process generally involves the folding of the one or more elongate, ‘scaffold’ strands into a particular shape using a plurality of rationally designed ‘staple’ oligonucleotide strands. The scaffold strand can have any sufficiently non-repetitive sequence. The sequences of the staple strands are designed such that they include sequences that hybridize to particular defined portions, or regions, of the scaffold strands and, in doing so, these two components cooperatively force the scaffold strands to assume a particular structural configuration. Staple strands are typically made from DNA but may also comprise RNA. Methods useful in the making of DNA origami structures can be found, for example, in Rothemund, P. W., Nature 440:297-302 (2006); Douglas et al, Nature 459:414-418 (2009); Dietz et al, Science 325:725-730 (2009); and U.S. Pat. App. Pub. Nos. 2007/0117109, 2008/0287668, 2010/0069621 and 2010/0216978, each of which is incorporated by reference in its entirety. Staple sequence design can be facilitated using, for example, CaDNAno software, available at http://www.cadnano.org or the DAEDALUS online platform, available at http://daedalus-dna-origami.org.

In embodiments of the invention the staple and/or scaffold components further comprise a plurality of hydrophobic membrane anchor molecules that are attached thereto. The hydrophobic anchors (or portions of the sequence) facilitate association of the nanostructure with a cellular membrane. Uptake of the nanostructures by the cell may occur through endocytosis or other similar mechanisms for internalisation of the plasma membrane by the cell. Typically individual nanostructures of the present invention will conform to dimensions that are less than 500 microns in size so do not require phagocytotic mechanisms unless there is substantial aggregation of the nanostructures on or close to the cell surface to the extent that larger agglomerated particles are formed. Phagocytosis is the mechanism by which cells take up larger particles into cell-surface membrane deformations which are internalised and processed in phagosomes. In contrast, endocytic processes are more appropriate for the smaller nanoscale structures described herein and may involve a range of mechanisms such as, but not limited to, clathrin-meditated and/or caveolar endocytosis. Once internalised within the cell, the nanostructures typically accumulate within intracellular vesicles such as lysosomes where they are processed. Without wishing to be bound by theory, it is currently believed that nucleic acids, such as mRNA, internalised within lysomal structures may escape to the cytosol if present in sufficient concentration; whereupon it mimics the endogenous mRNA and recruits ribosomes that enable translation of the ORF and, thus, biosynthesis of the encoded gene product within the cell.

In embodiments, the nanostructures of the present invention are formed or constructed from one or more modules. In embodiments, the nanostructure may be formed of an arrangement of modules that forms a basic frame or framework. In embodiments, the modules of the frame are supported by additional, typically smaller, sub-modules that connect and support the structure of the frame. In embodiments of the present invention the modules and sub-modules may comprise a plurality of substantially similar scaffold and staple nucleic acid structures that are assembled in the same way, and which associate to form a repeating structural motif.

The individual modules may be joined by nucleic acid strands, suitably DNA, the DNA strand either being integral with the module, or hybridised to each module. While any arrangement of the modules is contemplated, suitably, the modules may be arranged to form a range of nanostructures having a polygonal cross-section, the modules are arranged such that they sit side by side thereby defining the geometric configuration of the overall nanostructure. The modules may have tuneable side length (a side length in this context being defined as the longest dimension of the module), which when chosen with an appropriate final overall shape, allows for different sized and/or shaped nanostructures to be prepared. For example, the nanostructures defined by the assembly of modules may include a range of two and three dimensional geometric shapes, suitably selected from regular or irregular polyhedrons, with a cross section defining annular or solid shapes such as a circle, a triangle, a quadrilateral (e.g. a square, a rectangle or a trapezoid), a pentagon, a hexagon, a heptagon, an octagon and so on. Hence, nanostructures of the present invention may comprise spheroids, pyramidal shapes, cubes, or other polyhedral geometric shapes. As disclosed herein, in specific embodiments, the nanostructures comprise fully closed or partially closed nanobarrels, helical bundles, square plates and cuboids. However, it will be appreciated that the geometry of the nanostructure may be selected to accommodate a range of factors. These factors may be dependent upon inherent properties of the cargo nucleic acid sequence, such as its length; or may be defined by biocompatibility and biostability factors such as resistance to enzymic degradation, prevention of agglomeration, improved uptake by target cells etc.

Typically, a side length or maximum diameter of the nanostructures, or modules that are comprised within the nanostructures, is in the order of between 10 nm and 50 nm. Suitably, the side length of the modules may be at least 2 nm, 3 nm, 4 nm, 5 nm, 6 nm, 7 nm, 8 nm, 9 nm or 10 nm, Suitably the side length of the modules may be at most around 100 nm, 50 nm, 40 nm, nm, 20 nm and 10 nm. The sizing of sub-modules is determined by the spacing between the modules which is turn is determined by the shape of the nanostructure and the size and number of modules employed. Suitably, the side length of a module may be at least 0.5 nm, 1 nm, 1.5 nm, 2 nm, 2.5 nm, 3 nm, 3.5 nm, 4 nm or 5 nm, Suitably the side length of a module may be at most 10 nm, 7.5 nm, or 5 nm.

According to specific embodiments of the present invention the nanostructure includes a cargo nucleic acid that comprises at least one ORF that encodes a protein or polypeptide gene product. Suitably the cargo nucleic acid is a mRNA sequence that may be synthesised from a polynucleotide expression construct, which may be for example a DNA plasmid. This expression construct may comprise any promoter sequence necessary for the initiation of transcription and a corresponding termination sequence, such that transcription of the mRNA construct can occur.

In embodiments of the invention, the staple strands are oligonucleotides that hybridise to one or more complimentary regions of the scaffold sequence. The staples may be comprised of DNA or RNA or analogues thereof. A plurality of staples cooperate to impart the higher order secondary structure in combination with the scaffold strand through defined Watson-Crick base pair hybridisation interactions that draw and contort the scaffold strand according to nucleic acid origami principles. In a specific embodiment, an mRNA scaffold hybridises with one or more staples that comprise unpaired nucleotide extensions at the 5′ and/or 3′ terminus. These unpaired extensions may be one or two nucleotides or up to a dozen and serve to facilitate the steric accessibility of the ribosome to the mRNA and help the unzipping of the staple strands and subsequent translation of the scaffold mRNA.

In embodiments of the present invention the nanostructure comprises an mRNA that encodes at least one gene product that that increases an inflammatory or immunogenic immune response. This response may be mediated by, for example, delivery of a gene product that comprises an antigen. In such an instance, the nanostructure may act as a vaccine. Alternatively, the immunostimulatory response may be mediated by, for example, delivery of an immunomodulatory agent. Examples of such agents include antibody or antigen binding fragments thereof, or aptamers that bind to and inhibit immune checkpoint receptors (e.g. CTLA4, LAG3, PD1, PDL1, and others). A further immunomodulatory agent may comprise a proinflammatory cytokine such as IFNγ, IFNα, IFNβ, IL-6 or TNFα.

When utilised in vaccine-based therapies, the compositions and molecules defined in embodiments of the invention function as a novel vaccine form for use against infectious pathogens, such as viruses, bacteria, fungi, protozoa, prions, and helminths (worms); or for use in treating diseases such as cancer. Delivery of vaccine antigens within the nanostructures described can be used to induce a local immune response in a subject, or in order to provoke an adaptive immune response to the antigen itself—that is, to induce immunity against that antigen, similar to a vaccine. In such cases, the compositions according to the invention may be further combined with adjuvants to encourage the generation of a longer more pervasive immune response. Typically, suitable antigens for vaccine selection may include virion surface proteins, including surface exposed spike proteins that are involved in viral entry processes and/or cell receptor binding and membrane fusion.

As used herein; nanostructures comprising mRNA that encodes SARS-CoV-2 spike protein have been shown to be potentially effective as an RNA-based vaccine against COVID-19 disease. Hence, in some embodiments, the coding mRNA encodes all or part of any variant of the spike protein of the SARS-CoV-2. In some embodiments, the mRNA encodes all or a part of the spike protein's Receptor Binding Domain or RBD (e.g. residues 319 to 591; GenBank MN908947). Accordingly, the compositions of the present invention may be used in the prophylaxis or treatment of infectious pathogenic disease, such as by way of inclusion within vaccine formulations. Vaccine compositions and methods as discussed herein are non-exclusively contemplated for the treatment and prevention of diseases which may already be known to be susceptible to vaccination, particularly where an effective immunogenic protein is known. One of skill in the art could readily obtain similar antigens/targets from public databases and publications and generate compositions of the invention. By way of example, in some embodiments, the coding mRNA can encode one or more immunogenic viral proteins of the influenza virus (type A and B that cause epidemic seasonal flu). Multiple antigens may also be provided by the same, or different nanostructures as described herein, thereby allowing for a multivalent vaccine composition to be delivered to a subject in need thereof.

Hence, in embodiments of the present invention compositions may be formulated for use as vaccines that comprises nanostructures that include an ORF that encodes antigens that may include but are not limited to:
human cytomegalovirus—glycoprotein B, PP65 and/or IE1
hepatitis C (HCV)—E1 and E2 proteins
human immunodeficiency virus (HIV)—Gag and envelope proteins
Respiratory syncytial virus (RSV)—F protein
Ebola virus—EBOV glycoprotein
Tuberculosis—ESAT-6 and H37Rv proteins
Malaria—circumsporozoite proteins and derivatives such as VMP001

In addition to the aforementioned conventional preventive or prophylactic vaccine compositions, the invention may further provides for nanostructures that function as therapeutic vaccines which aim to provoke an immune response against targets which are already present in a subject's body, for example, against persistent infections or cancer. Hence, in one embodiment, nanostructures as described herein comprise nucleic acid cargo coding for tumoral antigen, for translation in tumour cells. This aims to induce an immune response against the cancer cells.

As described in more detail below, human cancer cells, such as HeLa cells, will readily take up nanostructures comprising a mRNA sequence that encodes a gene product and express the protein for a prolonged period of time (in this case eGFP). Cancer treatment vaccines are used typically in patients which are already diagnosed with cancer. The therapy can destroy cancer cells, stop tumour growth and spreading, or prevent the cancer from coming back after other treatments have ended. Suitably, the cancer vaccination strategy may involve selecting an appropriate nanostructure to deliver an mRNA encoding a tumour-associated neoantigen to the main antigen presentation cells of a subject's immune system, e.g. dendritic cells, which are then able to generate a long lasting anti-tumoral immune response. By way of example, Wilms tumour 1 (WT1) protein is an antigen that is detected in a wide range of malignancies and is a promising candidate for vaccine development.

Hence, a particular advantage of the nanostructures of the present invention is that they provide the opportunity to rapidly develop and test in vitro and in vivo whether a novel antigen is capable of functioning as a successful vaccine candidate whether of a cancer neoantigen or a for new or previously intractable pathogen.

Nanostructures of the invention may find utility is research and development, or diagnostics where introduction of a biomarker, such as a fluorescent protein, into a cell is needed. Suitable examples of intracellular marker probe proteins that may serve as cargo include green fluorescent protein (GFP) and homologues or derivatives thereof, such as enhanced GFP (eGFP), blue fluorescent protein (BFP, Azurite, mKalamal), cyan fluorescent protein (CFP, CyPet), yellow fluorescent protein (TFP, Citrine) and mCherry.

When administered to a subject, a therapeutic component that comprises the nanostructures of the invention is suitably administered as part of an in vivo delivery composition and may further comprise a pharmaceutically acceptable vehicle in order to create a pharmaceutical composition. The nanostructures may be formulated with a cationic compound which is able to complex with the negatively charged nucleic acid molecules and thereby to allow them to overcome the electrostatic repulsion of the cell membrane. Suitable cationic compounds may include one or more of: chitosan, polyethyleneimine (PEI), poly(2-hydroxy ethyl methacrylate) (pHEMA), polyamidoamine (PAMAM) dendrimers, polylactic-co-glycolic acid (PLGA), polyethylene glycol (PEG), poly-L-lysine (PLL), DOSPA (2,3-dioleoyloxy-N-[2(sperminecarboxamido)ethyl]-N,N-dimethyl-1-propaniminium trifluoroacetate), and DOPE (1,2-Dioleoyl-sn-glycerophosphoethanolamine). The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. Pharmaceutical compositions, if desired, can also contain minor amounts of wetting or emulsifying agents, or buffering agents.

The medicaments and pharmaceutical compositions of the invention can take the form of liquids, solutions, suspensions, gels, modified-release formulations (such as slow or sustained-release), or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

For any nanostructure or composition described herein, the therapeutically or diagnostically effective amount can be initially determined from in vitro cell culture assays. Target concentrations will be those concentrations of active component(s) that are capable of achieving the methods described herein, as measured using the methods described herein or known in the art.

As is well known in the art, therapeutically effective amounts of the compositions of the invention for use in human subjects can also be determined from animal models. For example, a dose suitable for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The invention is further illustrated by the following non-limiting examples.

### EXAMPLES

### Example 1—DNA Nanodevices with Selective Immune Cell Interaction and Function

To date, DNA nanotechnology has not yet been applied to improve unstructured mRNA or DNA delivery platforms in terms of low stability or selective interaction. Stability may be inferred by compacting nucleic acids into a nanostructure of higher stability and extended shelf-life. DNA nanostructures are also well known to present functional tags at defined positions, something which may be exploited to attain the desired functional interaction with immune cells. Any progress and insight on how DNA or RNA nanostructures interact with immune cells is hence of great interest.

DNA nanostructures have so far been tested in vivo using mouse models to determine their stability, biodistribution, and uptake-kinetics. Yet, several fundamental questions on the immune-relevant cellular interaction of DNA structure remain. Is it possible to achieve selective interaction of DNA nanostructure with immune cells in complex multicellular environments? Furthermore, what is the interaction with primary cells? Current studies focus mostly on adding DNA structures to cultured cell lines. If selective interaction is attained, do cell-bound nanostructures affect cellular membrane function and viability?

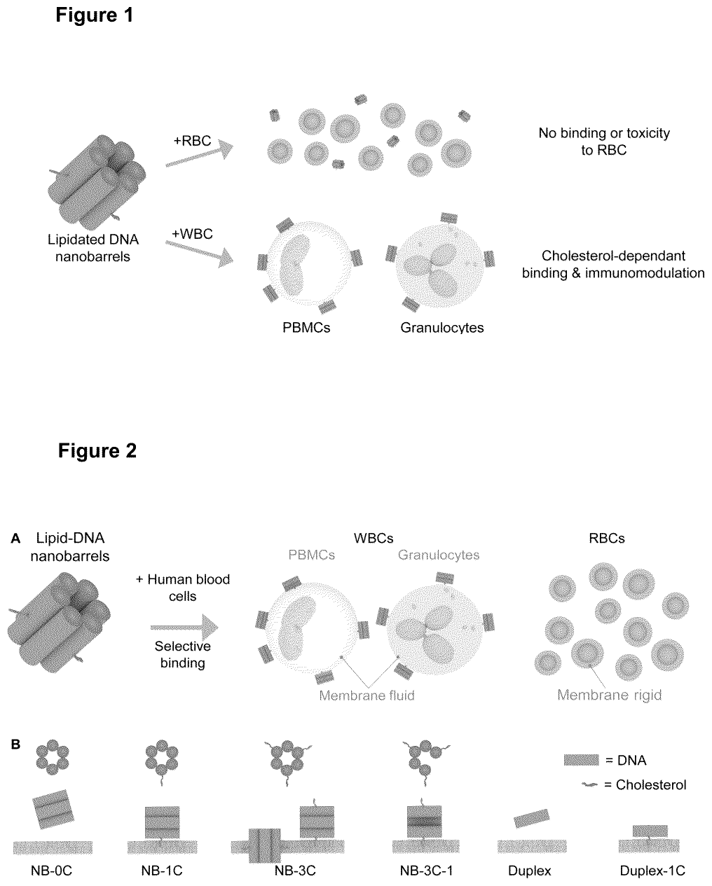

Here it is demonstrated that DNA nanostructures show a striking 400-fold immune-cell selective interaction with white blood cells in multi-cellular environments of primary human blood cells (FIGS. 1 and 2A). Primary human blood cells were examined since intravenous injection into blood is a standard delivery route for nucleic acids-based vaccines. White blood cells (immune cells) are also key as their immune responses to DNA nanostructures paves the way for potential novel immunotherapies. Human blood is mainly composed of red blood cells (erythrocytes), and a smaller percentage of white blood cells (WBCs), the latter is a mixture of peripheral blood mononuclear cells (PBMCs) and granulocytes.

To achieve targeted interaction with white blood cells, a new strategy was developed which exploits the differences in membrane composition between the target white blood cells and non-targeted red blood cells. In particular, white blood cells have a lower content of cholesterol and hence higher membrane fluidity. It was surmised that DNA structures with a cholesterol-tag would preferentially interact with the more fluid white blood cell membranes. A model DNA nanostructure in the form of a compact sub-10 nm-sized nanobarrel composed of a bundle of six interlinked duplexes (FIG. 2) was used. The DNA nanobarrel carries up to three cholesterol lipid anchors (FIGS. 2A, 2B). These have been shown to mediate binding to synthetic bilayer membranes but their ability to select for cell types is unknown. Our study shows a 400-fold selective binding of cholesterol-DNA nanobarrels to white blood cells over erythrocytes. The membrane-bound barrels do not cause any detectable puncturing or disruption to the cell membrane, thereby leaving cell viability unaffected. Selective barrels, strikingly, functionally modulate the immune cells by significantly suppressing the immune response towards an endotoxin (lipopolysaccharide) while being stable in all tested biological media. Here it is shown that a DNA device with designed selectivity advances the fundamental capabilities of DNA nanostructures in complex multicellular environments.

Results/Discussion

Structure of DNA Nanobarrels with Up to Three Cholesterol Anchors.

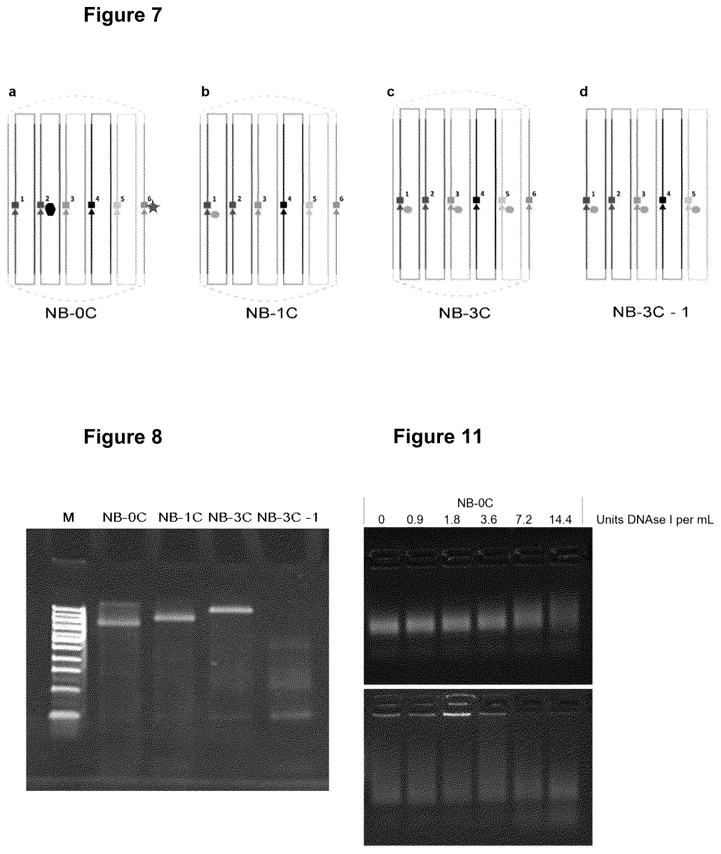

To test that DNA devices can selectively interact with blood cell membranes, DNA nanobarrels were used composed of six DNA duplexes which are interlinked to form a six-helical bundle measuring 9×5×5 nm (FIG. 2A, 2B, FIG. 7, Tables 1 and 2). Each hollow barrel has an inner lumen with a diameter of 2 nm. Three DNA nanobarrel constructs were examined. NB-3C, NB-1C, and NB-0C which contain 3, 1 and 0 cholesterol lipid anchors, respectively. The cholesterol anchors, which are designed to mediate the selective binding to white blood cells, are positioned, in variant NB-3C, symmetrically around the 6 helical barrel to aid membrane spanning behavior. This construct has been shown to porate synthetic bilayers upon successful membrane insertion to enable ion transport. The other two constructs serve as negative controls. NB-1C can tether but not span membranes, and NB-OC should not bind to any cellular bilayers (FIG. 2B). Additionally, a structurally incomplete NB-3C-1 construct containing 3 lipid anchors but lacking one unmodified strand was assayed to probe the influence of the barrel structure. Finally, single and double stranded oligonucleotides were used to determine the effect of linear DNA against assembled DNA barrels (FIG. 2B, Tables 1 and 2).

TABLE 1
Names, chemical modifications, and sequences
of DNA oligonucleotides used to prepare DNA
nanostructures.

| SEQ | |
|---|---|
| ID | |
| NO: | Sequence 5′ → 3′ |
| 1 | AGCGAACGTGGATTTTGTCCGACATCGGCAAGCTCCC |
| | TTTTTCGACTATT |
| 2 | CCGATGTCGGACTTTTACACGATCTTCGCCTGCTGGG |
| | TTTTGGGAGCTTG |
| 3 | CGAAGATCGTGTTTTTCCACAGTTGATTGCCCTTCAC |
| | TTTTCCCAGCAGG |
| 4 | AATCAACTGTGGTTTTTCTCACTGGTGATTAGAATGC |
| | TTTTGTGAAGGGC |
| 5 | TCACCAGTGAGATTTTTGTCGTACCAGGTGCATGGAT |
| | TTTTGCATTCTAA |
| 6 | CCTGGTACGACATTTTTCCACGTTCGCTAATAGTCGA |
| | TTTTATCCATGCA |
| 1 | Sequence of 1, carries a cholesterol |
| (chol) | via a tri(ethylene glycol) linker at |
| | the 3′ end |
| 3 | Sequence of 3, carries a cholesterol |
| (chol) | via a TEG linker at the 3′ terminus |
| 5 | Sequence of 5 carries a cholesterol |
| (chol) | via a TEG linker at the 3′ terminus |

For FRET assays, strand 2 contained a FAM dye, while strand 6 featured a TAMRA dye (see FIG. 7). Both dyes were at the 5′ terminus of the respective oligonucleotides. For CLSM a Cy3 or A647 dye was incorporated into strand 2, whilst for FACs a Cy5 dye was incorporated into strand 2.

TABLE 2
Names and composition of DNA nanobarrels
and control nanostructures

| Nano- | Oligonucleotides |
|---|---|
| structure | (SEQ ID NOs) |
| NB-0C | 1, 2, 3, 4, 5, 6 |
| NB-1C | 1(chol), 2, 3, 4, 5, 6 |
| NB-3C | 1(chol), 2, 3(chol), 4, 5(chol), 6 |
| NB-3C-1 | 1(chol), 2, 3(chol), 4, 5(chol) |
| SS | 1 |
| DS | 1, 2 |
| SS-1C | 1(chol) |
| DS-1C | 1(chol), 2 |

DNA Nanobarrel Assembly and Characterization.

The DNA nanobarrels were self-assembled by mixing equimolar ratios of each component oligonucleotide, followed by thermal annealing (FIG. 7, Tables 1, 2). Successful nanobarrel assembly in biocompatible PBS buffer was confirmed using polyacrylamide gel electrophoresis (FIG. 8). The assembled structures migrated with well-defined bands suggesting homogeneously folded products. Increasing the cholesterol number within the nanobarrel resulted in upshifted gel-band mobilities.

DNA Nanobarrels are Stable in Biological Media.

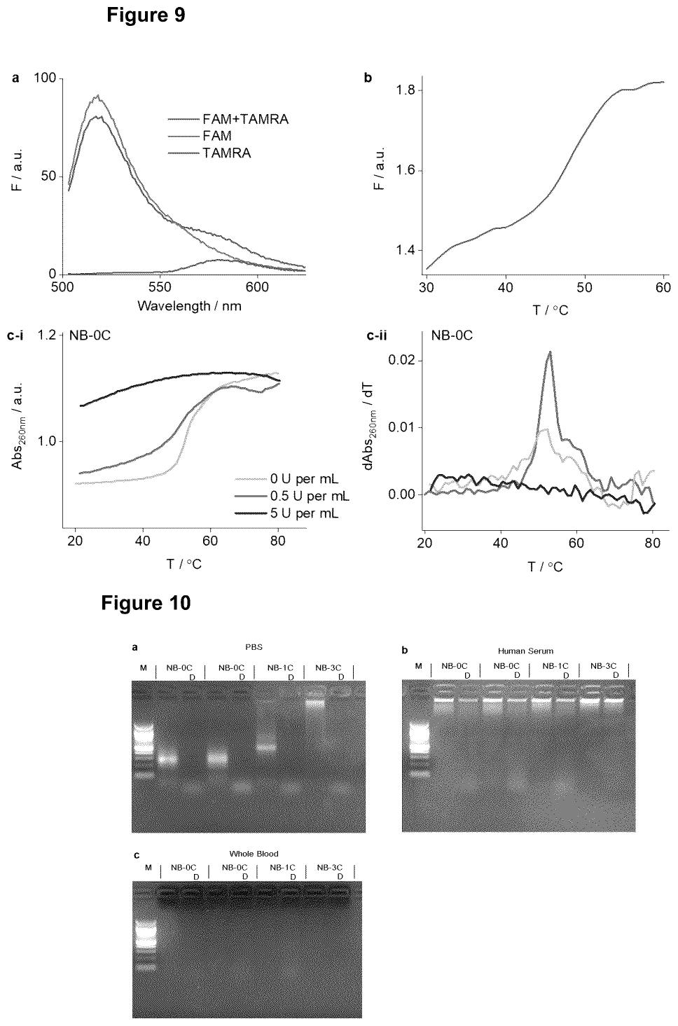

The structural stability of the DNA nanobarrels in biological media was examined with a temperature-induced unfolding assay. The melting temperature (Tₘ) of the DNA nanostructures was determined by monitoring fluorescence resonance energy transfer (FRET) upon heating. The FRET reporter dyes (fluorescein donor and TAMRA acceptor) were successfully incorporated into the DNA nanostructures (FIG. 7). At temperatures below the Tₘ, the nanobarrels are assembled and the attached FRET pairs are held in close contact, resulting in a high FRET efficiency with corresponding low donor fluorescence. At temperatures above the melting transition, the nanobarrels' strands dissociate leading to increased donor-acceptor distances, and higher donor emission (FIG. 9).

The nanobarrels' Tₘ transition was determined for NB-OC and NB-3C in a range of biological media (Table 3). The buffer systems assayed included PBS (nanobarrel folding buffer), Hank's buffered saline (HBSS), heat-inactivated human serum (HIS), human serum (HS) and whole blood (WB). In all media, the Tₘ values were higher or equal to PBS (Table 3, time point 0 h; FIG. 8), suggesting that the DNA nanobarrels remain stable under these diverse conditions.

TABLE 3
FRET-derived melting temperatures (Tm) of DNA
nanobarrels in biologically relevant media

| Construct | PBS | HBSS | HIS | HS | WB |
|---|---|---|---|---|---|
| NB-0C 0 | 46.2.7 ± 0.6 | 48.0 ± 2.0 | 47.2 ± 1.2 | 46.8 ± 1.1 | 46.2 ± 0.3 |
| NB-0C 24 h | 49.2.3 ± 0.3 | 48.8 ± 0.3 | 48.3 ± 0.3 | 48.3 ± 0.3 | 47.3 ± 0.3 |
| NB-3C 0 | 47.0 ± 0.0 | 47.0 ± 0.0 | 47.7 ± 0.3 | 47.7 ± 0.3 | 47.8 ± 0.3 |
| NB-3C 24 h | 48.3 ± 0.8 | 49.7 ± 0.3 | 48.5 ± 0.5 | 50.2 ± 0.3 | 48.7 ± 0.6 |

DNA nanobarrels assayed at 0.1 μM. Media include: phosphate buffered saline (PBS), Hank's buffered saline (HBSS), heat-inactivated human serum (HIS), human serum (HS) and whole blood (WB). Values are averages from 3 independent repeats, performed at either 0 or 24 h of sample incubation in the stated media.

The melting analysis also helped probe if the nanobarrels are resilient against DNA nucleases found in whole blood and human serum. Possible enzymatic degradation was determined by incubating the DNA barrels for 24 h and then measuring the melting profile. The Tm values showed no change (Table 3, time point 24 h) implying the DNA barrels are resilient to DNases found in these two bodily fluids for at least 24 h. These data were supported by gel electrophoretic analysis (FIGS. 10 and 11).

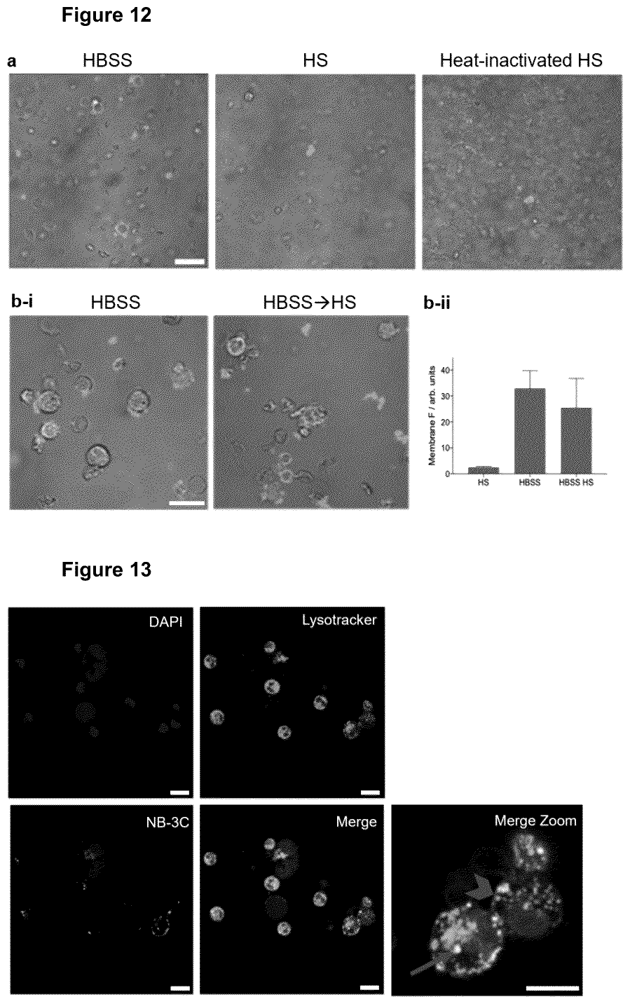

Nanobarrels were furthermore examined for possible aggregation using confocal laser scanning microscopy (CLSM). Previous studies have indicated that cholesterol-tagged DNA barrels are soluble in protein-free buffers but aggregate in the presence of serum proteins. Using a dye-labelled NB-3C barrel, no aggregation was observed in PBS or HBSS, but micron-sized aggregates occurred in human serum (FIG. 12). The large aggregates were due to the cholesterol anchors since NB-OC did not form any visible aggregates in either HBSS or human serum (data not shown). The subsequent experiments were conducted in HBSS to prevent aggregation.

DNA Nanobarrels Bind Selectively to White Blood Cells Via Cholesterol Anchors.

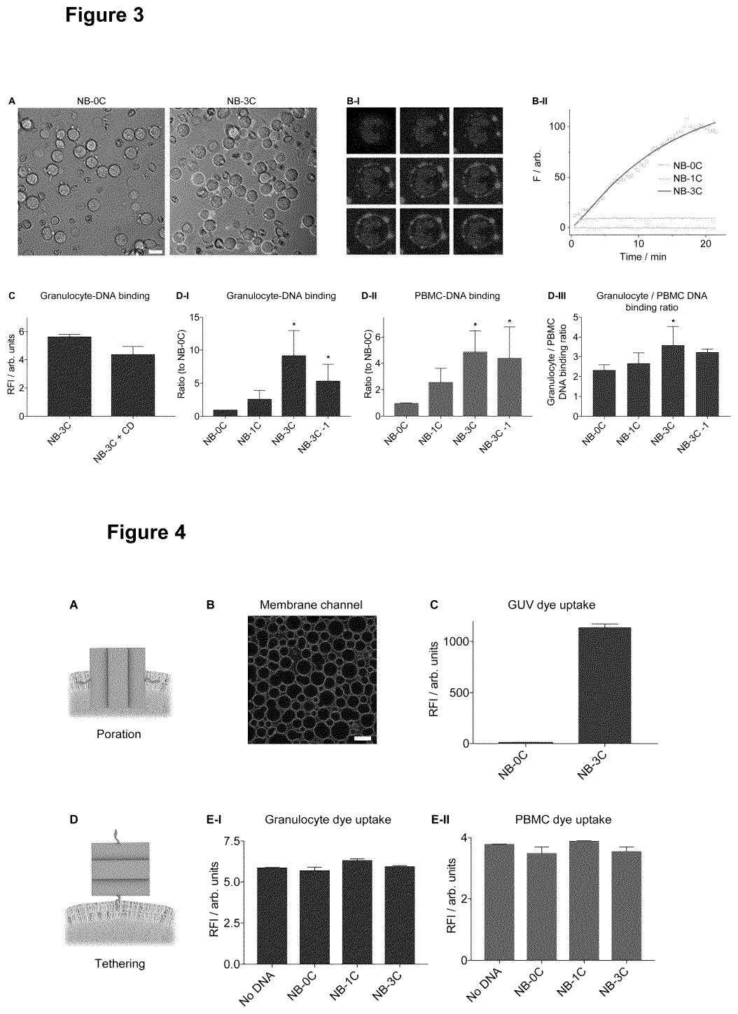

The interaction of nanobarrels with human blood cells was investigated to test whether the cholesterol lipid anchors can mediate selective interaction with white blood cells. Fluorescently labelled versions of DNA barrels NB-0C, NB-1C, and NB-3C (500 nM) were incubated with blood cells in HBSS. CLSM revealed fluorescent halos around white blood cells incubated with NB-3C (FIG. 3A) implying that the barrel tethers to the cell membrane. Cholesterol was required for cell membrane binding since NB-OC did not form halos (FIG. 3A). Microscopy further revealed that nanobarrels, strikingly, did not bind to red blood cells. NB-3C bound to white blood cells with over a, very large, 400-fold selectivity compared to erythrocytes (FIG. 3A), thereby validating the rational design.

The nevertheless strikingly high selective binding to WBCs occurred within a few minutes of nanobarrel addition (FIG. 3B-I). Three cholesterols were required for effective WBC binding as shown by the poor binding of NB-1C (FIG. 3B-II). Further analysis showed that cell-bound NB-3C remained on the surface of WBCs with minor cell-uptake. The limited internalized barrels co-localized with lysosomes (FIG. 13). The negligible internalization was confirmed by blocking phagocytosis with actin-inhibitor cytochalasin-D (CD) and flow cytometry (FIG. 3C). The observed minimal change in bound NB-3C (p=0.10) implies that the barrels remain on the membranes of non-blocked white blood cells.

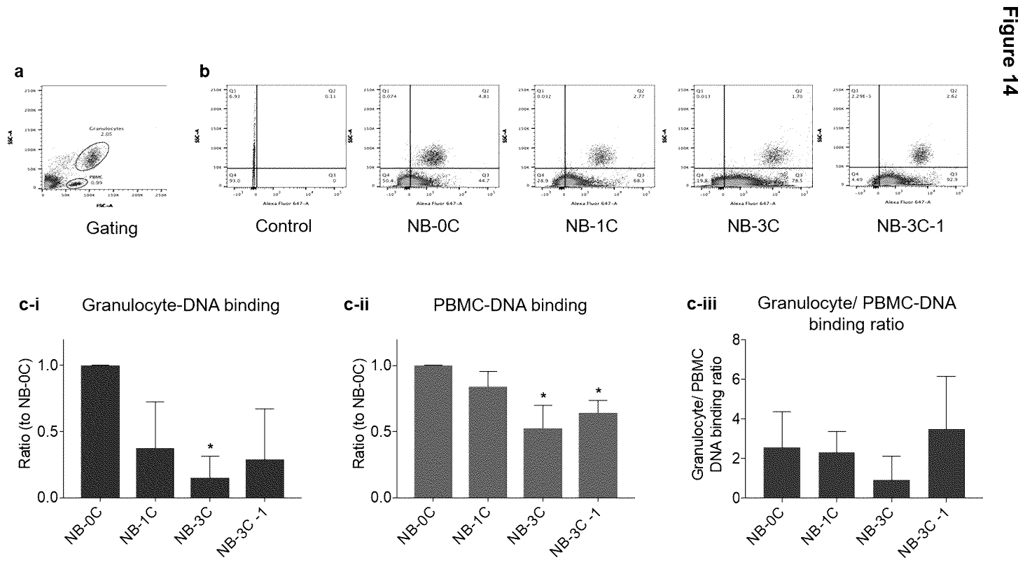

Flow cytometry was also used to further distinguish whether barrels bind preferentially to any of the specific white blood cell classes of granulocytes and peripheral blood mononuclear cells (PBMCs). Cells were incubated with healthy volunteer immune cells for 90 min with the A647-tagged DNA nanobarrel constructs (250 nM) and subjected to fluorescence activated cell sorting (FACS) (FIG. 14, FIG. 3D-I, 3D-II). The analysis revealed that nanobarrels bind weakly preferentially to granulocytes over PBMCs (FIG. 3D-III). For NB-3C, the binding was 3.6-fold stronger for granulocytes (p=0.004) (FIG. 3D-III). Furthermore, within each cell subset, binding increased with the barrels' cholesterol number (FIG. 3D-II, 3D-III). For example, NB-3C bound 9.2-fold more than NB-OC to granulocytes (p<0.001) and 4.9-fold more to PBMCs (p<0.001) (FIGS. 3D-I and 3D-II, respectively). The selective interaction with granulocytes could be due to differences in the membrane proteins on the PBMC but is nonetheless small when compared to the selectivity between white blood cells and erythrocytes.

NB-3C binding to WBCs was also compatible with human serum. In CLSM analysis, the fluorescent halo around WBCs was maintained when the cells were first incubated with NB-3C in HBSS for 5 min, and then transferred into human serum (FIG. 12). The fluorescence of the membrane-bound barrels was comparable in serum-free and human serum conditions indicating that serum proteins did not interfere with the interaction between the barrels and the WBCs. These results demonstrate that the DNA nanostructures bind highly selectively to WBCs over RBCs thereby raising the potential for selectively targeting and modulating immune cell responses.

DNA Nanobarrels do not Puncture White Blood Cell Membranes

It was evaluated whether barrel binding does or does not lead to membrane puncturing of granulocytes' and PBMCs' membranes or not. Cholesterol-tagged barrels have previously been shown to porate synthetic bilayers upon successful membrane insertion to enable ion transport. Any puncturing of cellular membranes would drastically lower the usefulness of nucleic acid nanostructures as delivery platform. Therefore, any possible puncturing of cell membranes was probed by monitoring passive transport of the water-soluble fluorescent dye Atto-647. The principle of the dye flux assay was first tested with giant unilamellar vesicles (GUVs) where the small cationic dye55 was expected to translocate through membrane-inserted nanobarrels (FIG. 4A).

In contrast to barrel-induced poration of synthetic bilayers, granulocytes and PBMCs did not display dye transport even after 6 h of incubation with NB-3C (FIG. 4D, 4E). This surprising finding suggests that the DNA barrel tethers side-on to the cell membrane (FIG. 4D) rather than spanning it (FIG. 4A). The desirable lack of barrel insertion into granulocytes and PBMC membranes may be due to steric hinderance by the densely packed proteins on the cell surface which is different to synthetic membrane systems.

DNA Nanobarrels do not Affect the Viability of White and Red Blood Cells

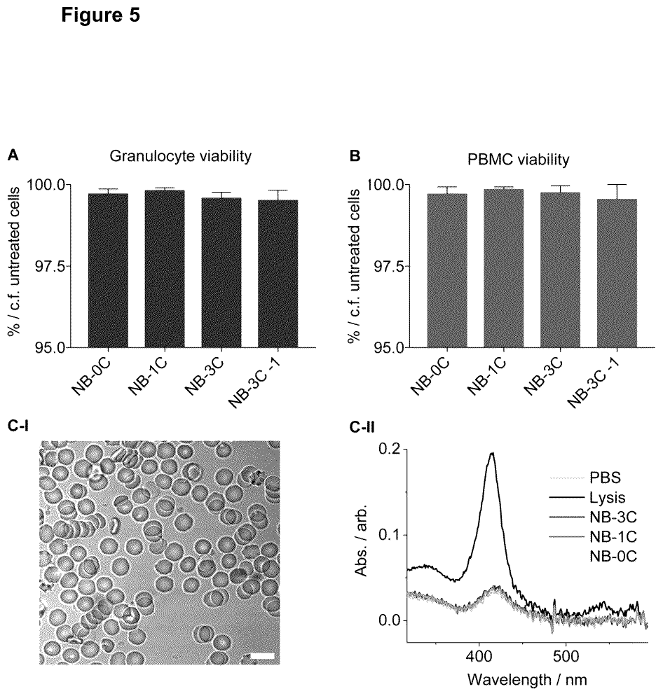

Having established selective and non-rupturing nanobarrel binding to white blood cells, we tested any effect on cell viability. As expected from their non-puncturing membrane binding, WBCs viability was unaffected after 6 h of nanobarrel incubation (FIG. 5A, 5B). The barrels were also non-toxic to RBCs. Neither NB-3C nor NB-OC bound to erythrocytes in CLSM analysis (FIG. 5C-I), and the cells' viability was not compromised after 4 h incubation using a haemolysis read-out (FIG. 5C-II).

DNA Nanobarrels Initiate a Differential Immune Response in White Blood Cells

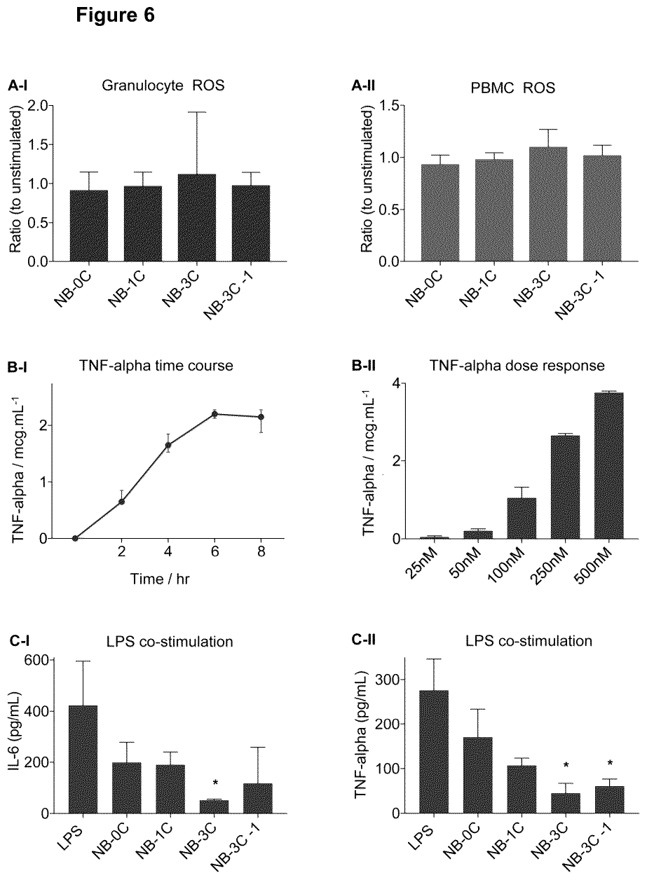

Having established the barrels' non-toxic and highly selective binding to white blood cells, it was next investigated whether DNA barrels trigger a change in immune function. DNA binding to other, cultured immune cells is known to lead to a proinflammatory reaction. It was probed whether the DNA barrels also behave in a similar way. To assess the white blood cells' immune response to DNA barrels, first the production of intracellular reactive oxygen species (ROS) was monitored using the free radical-sensitive fluorophore dihydroethidium. When WBCs were challenged with nanobarrels, no increase in ROS levels was detected in granulocytes (p=0.41) (FIG. 6A-I) or PBMCs (p=0.06) (FIG. 6A-II) after 90 min of incubation in HBSS.

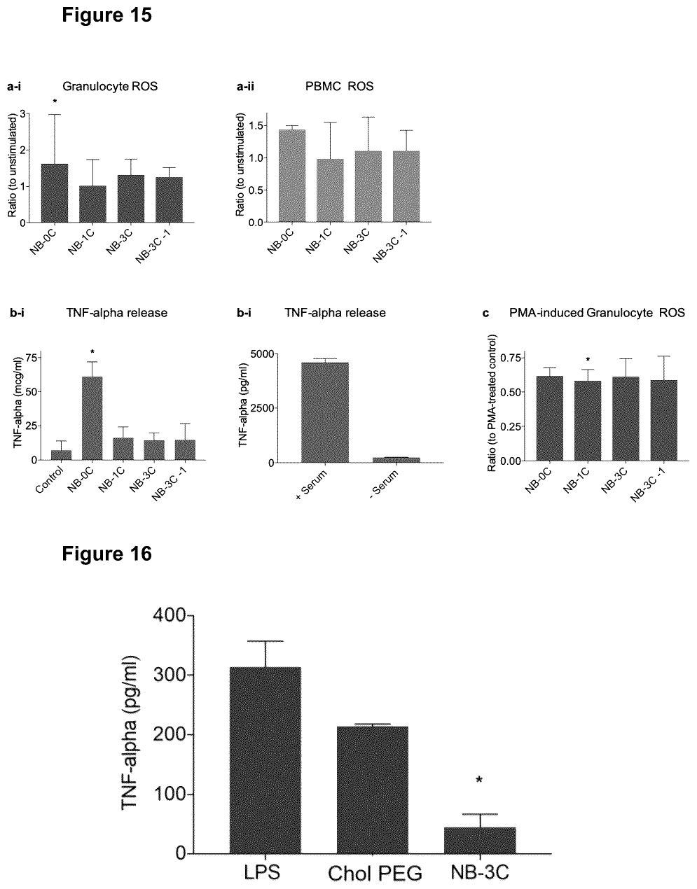

Similarly low levels of ROS were obtained when WBCs were challenged with DNA barrels in whole blood. Only a small 1.6-fold increase in ROS level was observed in granulocytes incubated with NB-OC (p=0.032) (FIG. 15). The absence of an intense ROS immune response is unexpected considering the strong binding of DNA barrels, and also because DNA strands usually produce a stronger immune response. The molecular reason for the observed lower response could lie in the highly compact nature of the DNA nanostructures which is different to the looser conformation of previously tested nanostructures with single or double stranded DNA appendages. Compacted DNA might not be a good substrate for the ROS-generating pathway.

To further gain insight into the immune response, the release of pro-inflammatory cytokines interleukin-6 (IL-6) and tumour necrosis factor-alpha (TNF-alpha) was monitored. Cytokines IL-6 or TNF-alpha levels were not elevated following 6 h incubation in HBSS (data not shown). Cytokine production was, however, significantly elevated in the presence of serum co-factors. For example, TNF-alpha levels were higher upon incubation in whole blood (p=0.002) (FIG. 6B). Under these conditions, the TNF-alpha level was proportional to the incubation duration up to 6 h (FIG. 6B-I) and the nanobarrel NB-3C concentration (FIG. 6B-II). The data on elevated cytokine production yet low ROS levels indicate that DNA barrels lead to a differential immune response.

Lipidated DNA Nanobarrels Modulate the Immune Response after Stimulation with Inflammatory Tumour Promotors and Endotoxins

After establishing the base-line response to the DNA nanostructures, the more complex topic of how the barrels alter the immune reaction to strong inflammatory stimuli was explored. In particular, an examination was carried out on how pre-incubation with barrels affects the granulocytes' reaction to an inflammatory stimulant and tumour promotor phorbol 12-myristate 13-acetate (PMA). Usually, PMA intracellularly activates the protein kinase C pathway resulting in a respiratory burst with increased production of ROS superoxides. Pre-incubation with DNA barrels for 90 min led, however, to a 40% lower production of ROS compared to non-pre-treatment (p=0.052)(FIG. 15). This surprising reduction was found for all types of DNA barrels implying a limited influence of the cholesterol anchors. The cholesterol-independent effect of DNA barrels may be rationalized by the molecular interaction of PMA which acts independently of cell surface receptors and should hence not directly compete with DNA barrel binding on the cell membrane.

It was also tested how pre-incubation alters the reaction to endotoxin lipopolysaccharide (LPS) which binds on the cell membrane's toll-like receptors (TLRs). Addition of the endotoxin usually results in a significant increase in cytokine release, as found when granulocytes were challenged with LPS (100 ng/mL) for 4 h (FIG. 6C-I, FIG. 15). However, the response was suppressed when immune cells were first incubated with DNA nanobarrels for 90 min followed by LPS. For example, pre-incubation by NB-3C significantly reduced the release of cytokine L-6 9.4-fold (p=0.01) and TNF-alpha levels 6.4-fold (p=0.007) (FIG. 5C-I, FIG. 16); LPS did not alter ROS (data not shown). The suppression of the cytokine response was dependent on the number of cholesterols, with NB-3C displaying the biggest change (p=0.02) (FIG. 6C-II). The cholesterol-dependence may be explained by the competitive binding to toll-like receptors of the DNA barrels and the equally negatively charged LPS. This endotoxin data underscores that DNA barrels can lead to a surprising immune suppression for two classes of inflammatory stimuli.

Conclusions

DNA nanotechnology produces highly defined materials that open up new strategies for biomedical applications. The present embodiments described substantially advance the field by revealing how DNA nanostructures behave in multi-cellular environments with impact in cellular targeting, cell membrane biology, and immunology including vaccine development. These findings are based on biologically important and biotechnologically representative components: blood cells and an antibody-sized DNA nanostructure equipped with lipid cholesterol tags.

This example delivers three fundamental insights. Firstly, it establishes a 400-fold preferential binding of the DNA nanobarrel structures to white blood cells compared to red blood cells (FIG. 1). The rapid, cellular binding only occurs with cholesterol labelled DNA structures. Previous studies on synthetic membrane bilayers did not investigate selective binding. The cellular targeting is attributed to the difference in lipid membrane composition of the two cell classes whereby WBCs membranes have a lower content of cholesterol than RBCs. In particular, white blood cells' lower content of cholesterol leads to higher membrane nanofluidity due to dynamic voids within the bilayer. Fluid membranes with voids can, following the present explanation of selectivity, be filled with cholesterol-tags of DNA barrels.

The impact of cholesterol-mediated selectivity may lie in the field of vaccine development and drug targeting. Cholesterol tags or other membranes-selective moieties may be used as a new route to target functional DNA or RNA cargo towards immune cells thereby complementing aptamer-based recognition. Vaccine development benefits from this selective interaction as nucleic acid vaccines have to be taken up by immune cells to translate the genetic information into a protein that elicits the immune response. Cholesterol-mediated binding may also be a general basis to assist targeting other biomedically relevant substances to immune cells, especially for immunomodulation. While this present data shows that cholesterol is a main source for selectivity, binding can also be modulated by membrane proteins. Protein interaction may, for example, explain the small 3-fold difference in binding between the two WBC subclasses of granulocytes and PBMCs which have similar cholesterol levels. Strategies to tackle aggregation of DNA barrels in serum, which is either due to electrostatic or hydrophobic interactions, may include masking negative charges using charge neutralized DNA or PNA, coating by proteins or PEG moieties, or placing cholesterol lipid anchors in less accessible, recessed positions along the DNA nanostructure.

As second insight, avid cholesterol-mediated binding to white blood cells did not lead to membrane rupturing. The non-toxic behaviour is beneficial for any biomedical applications, yet surprising as the cholesterol nanobarrels are known to porate synthetic membranes. The barrels are likely non-toxic to cells as one cholesterol tag is sufficient for membrane tethering, while complete membrane insertion with three cholesterols is potentially blocked by crowding-out from proteins on the membrane surface.

Thirdly, the membrane-bound DNA nanostructures were discovered to repress immune cell responses to endotoxin (FIG. 6). DNA usually activates the immune system and CpG repeat sequences have been shown to enhance this response. The present cholesterol-tagged DNA nanobarrels elicited the release of proinflammatory cytokines in a dose-dependent manner in serum. Surprisingly, DNA barrels reduced the cytokine release from leukocytes that were incubated with pro-inflammatory stimulus lipopolysaccharide (LPS). Without barrel pre-incubation, LPS led to the expected inflammatory cytokine release. A possible explanation for the immune-suppression rests on the negatively charged character of both DNA and LPS. It is hypothesized that DNA barrels may electrostatically block LPS engagement to the cognate surface toll-like receptors, thereby resulting in an attenuated downstream cascade activation and reduced immune response.

In conclusion, this example provides an account of the behaviour of a series of compact DNA nanostructures in a multicellular environment with biomedical impact for potential therapeutic applications. Promising routes include the development of vaccines to deliver compact and nuclease-resistant DNA to target immune cells. In addition, in immunomodulatory therapy DNA structure binding could help tackle septic shock by decreasing the exaggerated host response to infection as supported by the abrogated LPS response of immune cells. Other applications of attenuated immune cell response include transplant immunosuppression and autoimmune disease.

### Methods/Experimental Section

Reagents. All other reagents were purchased from Sigma Aldrich (UK) unless stated.

Folding of DNA nanostructures. The structure of the DNA nanobarrel is as set out in Burns et al. Nat. Nanotechnol. 2016, 11, 152-156. The DNA nanobarrels were assembled by mixing an equimolar mixture of the component DNA strands (1 μM) (Integrated DNA Technologies, US) containing the stated buffer or media. The constructs were folded by heating the solution from for 2 min, and cooling to 20° C. at a rate of 5° C. per min. The assembled constructs were stored at room temperature and vortexed for 2 s before use and used within 24 h.

Polyacrylamide gel electrophoretic analysis of folded DNA nanostructures. The DNA nanobarrels (2 μL, 1 μM in PBS) were added to buffer (13 μL, PBS unless otherwise specified) and gel loading dye (5 μL, purple loading dye). Each sample (8 μL) was loaded into wells of a thermally equilibrated PAGE gel (10% in 1×TBE buffer for native or 1×TGS buffer for SDS PAGE, at 115 V for 60 min at 4° C.). Once complete, the bands were visualized by first washing in de-ionized water, and then stained with ethidium bromide solution.

Agarose gel electrophoresis. The DNA nanobarrels (2 μL, 1 μM in PBS) were added to buffer (13 μL, PBS) and gel loading dye (5 μL, SDS-free). The solution (15 μL) was loaded into the wells of a thermally equilibrated agarose gel (2% in 1×TAE buffer, containing ethidium bromide), and run at 60 V for 60 min at 4° C.

Whole blood and serum preparation. Whole blood (5 mL) was collected in heparinized syringes from healthy donors. Cells were separated from plasma by centrifugation for 15 min at 2,000×g using a refrigerated centrifuge. The resulting supernatant containing plasma was extracted and used immediately or stored at −20° C. prior to further use. Repeated freeze thaw cycles were avoided. To heat-inactivate human serum, the media was incubated at 60° C. for 20 min.

Nanostructure stability assay with fluorescence resonance energy transfer. The melting transitions of the DNA nanostructures were identified following a published procedure set out in Burns et al. Nanomaterials 2019, 9, 490 using a MyIQ real-time PCR (Bio-Rad, Watford, UK). The nanostructures were assembled containing FAM and TAMRA FRET pairs (folded at 1 μM in PBS). The DNA constructs were diluted into the stated buffer systems to give a final DNA concentration of 0.1 μM (total volume of 25 μL) in a 96-well thin wall fluorescence plate (Bio-Rad, Watford, UK). Optical quality sealing tape (Bio-Rad, Watford, UK) was placed on top to prevent evaporation. The samples were heated from 30 to 85° C. at a rate of 0.5° C. per min. The melting temperature was determined from taking the 1st derivative of the donor fluorescence profile. Errors were identified from three independent experiments.

Isolation of white blood cells for confocal laser scanning microscopy and flow cytometry. Whole blood (5 to 10 mL) was collected in heparinized syringes from healthy donors. RBCs were lysed using 1× red cell lysis buffer (BD; Beckton Dickinson biosciences, UK) containing 16% formaldehyde. Cells were washed and re-suspended in either human serum (5 mL) or HBSS (5 mL), for serum and serum-free conditions, respectively. The nuclear stain 4′,6-diamidino-2-phenylindole (DAPI) (Thermo Fisher Scientific, UK) at 5 μg per mL was added to visualize WBCs for confocal microscopy and flow cytometry.

Confocal laser scanning microscopy. CLSM images were collected using a 60× oil objective mounted on a FV-1000 Olympus microscope, solutions were deposited on a fluorodish (World Precision Instruments, Sarasota, FL, USA) and left to settle for 5 min before imaging. For serum stability assays, the DNA solution (20 μL, 1 μM in PBS) was added to either HBSS, human serum or heat-inactivated human serum (20 μL) and mixed for 5 mins. Where stated WBCs (2 μL) were additionally added and deposited on the confocal dish. For kinetic WBC binding assays, WBCs (in HBSS or human serum, 10 μL) were added to a confocal dish and left to settle for 5 mins, followed by addition of the DNA solution (10 μL, 1 μM in PBS). All imaging conditions were kept identical when comparing different DNA barrel constructs and controls. Images were analysed using ImageJ software.

Fluorescence activated cell sorting. Cells were analysed on the LSR Fortessa (BD) flow cytometer (BD Biosciences). The two main cell populations, granulocytes and peripheral blood mononuclear cells were identified by gating on characteristic forward and side scatter profiles. Identical gates were applied to all samples. Minimum of 5000 events/measurement within the granulocyte population were read. For binding assays, the DNA solutions (200 μL, 500 nM in PBS) were added to WBCs (in HBSS or HS, 200 μL) and incubated for 90 min. All data were collected from three individual replicates per experiment and geometric mean assessed using FlowJo version 10.0 (Tree Star Inc, USA). Statistical data analysed using a non-parametric Mann Whitney t-test using GraphPad Prism v5 (San Diego, USA).

White blood cell viability. The DNA solutions (1 mL, 500 nM in PBS) were added to WBCs (in HBSS, 1 mL) and incubated for 6 h. Viability of cells was determined by the addition of far red live/dead stain (Thermo Fisher Scientific, UK) 20 min prior to fixation with formaldehyde at 37° C. As a positive control for dead cells, cells were incubated at 60° C. for 10 min.

Haemolysis. The DNA nanobarrels (20 μL, 1 μM in PBS) were added to RBCs (in HS or HBSS μL) and the solution mixed for 4 h. The extent of lysis was determined by diluting the solution (15 μL) in PBS (600 μL) and monitoring the UV-vis absorbance. Experiments were performed in triplicate.

White blood cell activation. Immune cell reactive oxygen species (ROS) was determined by preincubating blood (in HBSS, 2 mL) with dihydroethidium (5 μM) or H2-DCFDA (5 μM) (Thermo Fisher Scientific, UK) for 30 min at 37° C. prior to the addition of the DNA constructs (2 mL, 500 nM in PBS) or other stimuli. Phorbol 12-myristate 13-acetate (PMA) and inomicin (eBioscience cell stimulation cocktail) was used to activate neutrophils either as a positive control for reactive oxygen species (ROS), or to assess PMN ROS capacity following incubation with DNA constructs. Lipopolysaccharide was obtained from Sigma and used at a concentration of 100 ng per mL.

ELISA. Release of TNF-alpha and IL-6 from immune cells in either whole blood or supernatants was measured using ELISA. The DNA constructs (5 mL, 500 nM in PBS) were incubated with whole blood (5 mL) up to 8 h. Whole blood samples were centrifuged, and plasma used for analysis of cytokine levels. DuoSet ELISA kits (R&D Systems, Minneapolis, MN, and BD Biosciences, Oxford, Oxon, UK) were used to assess cytokine levels according to the manufacturers' instructions. Absorbance was read at 450 nm using a spectrophotometric ELISA plate reader (Anthos Hill; Anthos Labtec, Salzburg, Austria).

### Example 2—DNA Nanostructures Provide Stable Delivery Platform for mRNA to Recipient Cells

Origami nanostructures composed of a mRNA scaffold strand and DNA oligonucleotide staple strands were designed. Using these structures the aim was to test whether the incorporation of the mRNA into an origami nanostructure would confer functional benefits to the mRNA domain. The functional advantages may include improved stability against nuclease digestion enzymes, repeated freeze and thaw cycles, and higher divalent metal anion stability when compared to the isolated mRNA.

For the scaffold strand an mRNA sequence was used that comprises an ORF which translates into green fluorescent protein (GFP). The mRNA is 720 nucleotides long and its sequence is shown below (SEQ ID NO: 7).

mRNA EGFP sequence (5′ to 3′)-.
SEQ ID NO: 7
AUGGUGAGCAAGGGCGAGGAGCUGUUCACCGGGGUGGUGCCCAUCCUGGU
CGAGCUGGACGGCGACGUAAACGGCCACAAGUUCAGCGUGUCCGGCGAGG
GCGAGGGCGAUGCCACCUACGGCAAGCUGACCCUGAAGUUCAUCUGCACC
ACCGGCAAGCUGCCCGUGCCCUGGCCCACCCUCGUGACCACCCUGACCUA
CGGCGUGCAGUGCUUCAGCCGCUACCCCGACCACAUGAAGCAGCACGACU
UCUUCAAGUCCGCCAUGCCCGAAGGCUACGUCCAGGAGCGCACCAUCUUC
UUCAAGGACGACGGCAACUACAAGACCCGCGCCGAGGUGAAGUUCGAGGG
CGACACCCUGGUGAACCGCAUCGAGCUGAAGGGCAUCGACUUCAAGGAGG
ACGGCAACAUCCUGGGGCACAAGCUGGAGUACAACUACAACAGCCACAAC
GUCUAUAUCAUGGCCGACAAGCAGAAGAACGGCAUCAAGGUGAACUUCAA
GAUCCGCCACAACAUCGAGGACGGCAGCGUGCAGCUCGCCGACCACUACC
AGCAGAACACCCCCAUCGGCGACGGCCCCGUGCUGCUGCCCGACAACCAC
UACCUGAGCACCCAGUCCGCCCUGAGCAAAGACCCCAACGAGAAGCGCGA
UCACAUGGUCCUGCUGGAGUUCGUGACCGCCGCCGGGAUCACUCUCGGCA
UGGACGAGCUGUACAAGUAA

The designed mRNA-DNA hybrid nanostructures are shown in FIG. 17 and set out in Tables 4 and 5 below. The first structure is the linear duplex (LD) composed of staple strands that bind along the mRNA sequence to form an elongated duplex rod (FIG. 17(B)). The helical bundle (HB) in which the mRNA and the staple strands form six duplexes which are arranged in hexagonal order when viewed along the axis of the duplexes. The six duplexes are interconnected via DNA origami cross-overs and hairpins (FIG. 17 (C)). In the square plate (SP), the mRNA and the staples are folded into 10 duplexes that are aligned parallel to form a planar unit (FIG. 17 (D)). In the cube (CB), the duplexes assemble to form a 3×3 cube-shaped bundle (FIG. 17 (E)).

The sequences of the staple strands are shown in Table 4 and the DNA staple composition of GFP-DNA hybrid constructs is shown in Table 5.

TABLE 4
DNA staples for GFP mRNA-DNA constructs

| | SEQ | |
|---|---|---|
| | ID | |
| Name | NO: | Sequences 5′ to 3′ |
| LD0[37] | 9 | ACCACCCCGGTGAACAGCTCCTCGCCCTTGCTCAC |
| | | CAT |
| LD0[387] | 10 | CGATGCCCTTCAGCTCGATGCGGTTCACCAGGGTG |
| LD0[72] | 11 | CGTTTACGTCGCCGTCCAGCTCGACCAGGATGGGC |
| LD0[107] | 12 | GCCCTCGCCCTCGCCGGACACGCTGAACTTGTGGC |
| LD0[422] | 13 | CTTGTGCCCCAGGATGTTGCCGTCCTCCTTGAAGT |
| LD0[667] | 14 | CCAGCAGGACCATGTGATCGCGCTTCTCGTTGGGG |
| LD0[457] | 15 | ATATAGACGTTGTGGCTGTTGTAGTTGTACTCCAG |
| LD0[142] | 16 | ATGAACTTCAGGGTCAGCTTGCCGTAGGTGGCATC |
| LD0[632] | 17 | TCTTTGCTCAGGGCGGACTTGGGTGCTCAGGTAGT |
| LD0[177] | 18 | TGGGCCAGGGCACGGGCAGCTTGCCGGTGGTGCAG |
| LD0[492] | 19 | TCACCTTGATGCCGTTCTTCTGCTTGTCGGCCATG |
| LD0[527] | 20 | GCTGCCGTCCTCGATGTTGTGGCGGATCTTGAAGT |
| LD0[212] | 21 | GCACTGCACGCCGTAGGTCAGGGTGGTCACGAGGG |
| LD0[247] | 22 | TCGTGCTGCTTCATGTGGTCGGGGTAGCGGCTGAA |
| LD0[562] | 23 | GGGGTGTTCTGCTGGTAGTGGTCGGCGAGCTGCAC |
| LD0[597] | 24 | GGTTGTCGGGCAGCAGCACGGGGCCGTCGCCGATG |
| LD0[702] | 25 | CATGCCGAGAGTGATCCCGGCGGCGGTCACGAACT |
| LD0[282] | 26 | GGACGTAGCCTTCGGGCATGGCGGACTTGAAGAAG |
| LD0[720] | 27 | TTACTTGTACAGCTCGTC |
| LD0[352] | 28 | TCGCCCTCGAACTTCACCTCGGCGCGGGTCTTGTA |
| LD0[317] | 29 | GTTGCCGTCGTCCTTGAAGAAGATGGTGCGCTCCT |
| HBC3[13] | 30 | TTTGATGAACTTCAGCCAGCTCGATTT |
| HBC4[138] | 31 | TTTTCTCAGGGCGGACTTGGCTCGTTGTTTTTTTT |
| | | TTT |
| HBC5[52] | 32 | TTTTCTGAACTATCGCCCTCGCCCTTTTTTTTTTT |
| HBC5[16] | 33 | TTTCCAGGATGGGCACCACTCGCCGTGGTCAGCTT |
| | | GCCGTTTT |
| HBC0[131] | 34 | TTTTGTGGGTCTTTGTTTT |
| HBC5[95] | 35 | TTTGGCAGCACACGCTGCCGTCCTGGCCATGTTTT |
| HBC2[72] | 36 | TTTTTTTTTTCGTTCTGCTGGTAGTGGTCGGTTTT |
| HBC1[9] | 37 | TTTTCATGTGGTCGGGGTAAAGAAGTCGTGCTGCT |
| | | TTT |
| HBC1[95] | 38 | TTTATATAGACGTTGTGCGAGCTGTTTTTTTTTTT |
| HBC4[71] | 39 | TTTTTTTTTTTTTTTTCTTGCTCGCTCCTGGACGT |
| | | AGTTTTTTTTTTTT |
| HBC1[61] | 40 | TTTTTTTTTATGGTGCACCATAGTGATCCCTTTTT |
| HBC0[114] | 41 | TTTTGCGGTTTTCTTCTGCTTGTCCGATGTTTTTT |
| HBC2[52] | 42 | TTTTTTTTTTTAGGTGGCTGTGGCCGTTTACGTTT |
| HBC2[134] | 43 | TTTTACCTTGATGCCGCACCAGGGTTTTT |
| HBC5[38] | 44 | TTTTGCATGGCGGACTTGGCGGCTGGCAGCTTGCC |
| | | GGTGGTGCATTT |
| HBC2[38] | 45 | TTTTAAGCACTGCACGCCCCTTCGGTTTTTTTTTT |
| HBC4[114] | 46 | TTTGTGGCGGATCTTGAAGTTCTTTT |
| HBC1[102] | 47 | TTTTTTTTTTACCTCGGATGTGATCGCGCTTTTTT |
| HBC2[79] | 48 | TTTGCCGTCCTCCTTGATTGCCGTTTTTTTTTTTT |
| HBC4[79] | 49 | TTTGGCGGCGCGTCCTTGAAGAAGGGATGTTTTTT |
| HBC4[107] | 50 | TTTTTTTTTTCAGGACCCGCGGGTCTTGTAGTTTT |
| SP8[23] | 51 | ACCACCCCGAACTTGTGGCCGTTTCCGTAGGT |
| SP4[23] | 52 | TGGTCACGATGTGGTCGGGGTAGCCTTCGGGC |
| SP4[37] | 53 | GCCGTAGATGAACTTCACGATGGTCCTGGGTC |
| SP0[23] | 54 | CTTCACCTCGGCGCGGGTCTTTT |
| SP6[72] | 55 | TTTTGTCGGTAGTGTTTT |
| SP3[1] | 56 | TTTTCGTGCTGCTTCAGGGTGGGCCATTTT |
| SP4[55] | 57 | TCTTCTGCTGATATAGACGTTGTGTTGTACTC |
| SP7[1] | 58 | TTTTCCGGACACGCTGGTGAACAGCTTTTT |
| SP4[72] | 59 | TTTTACCTTAGTTCTTTT |
| SP6[37] | 60 | AGCTTGACGTCGCCGTCCCGATCGTCGCAGCTCGA |
| | | CCACTTTGCTCA |
| SP2[23] | 61 | ATGGCGGAGTCGTCCTTGAAGAAGCCCTCGAA |
| SP2[72] | 62 | TTTTTGTAGGCTGTTTTT |
| SP9[1] | 63 | TTTTCCTCGCCCTTGCTCACCATGTTGGGGTGGAT |
| | | GGGC |
| SP0[55] | 64 | TCGATGCGGTTCACCAGGGTGTCGATGGTGCGCTC |
| | | AGGATGCCCCCTGGA |
| SP6[55] | 65 | ACGCTGCCTTGTGGCGGATCTTGAGATGCCGT |
| SP9[41] | 66 | GGGCGGACTTGGGTGCTCAGGTAGGCAGCAG |
| SP2[37] | 67 | CGTAGCGGCTGAAGCACGGCCATTGTCTGCAC |
| SP1[1] | 68 | TTTTTTGTAGTTGCCCTTGAAGAAGTTTTT |
| SP0[72] | 69 | TTTTGATGCAAGTCTTTT |
| SP6[23] | 70 | GGCATCGCGCTTGCCGGTGGTGCAGGTCAGGG |
| SP2[55] | 71 | CAGCTTGTGTTGCCGTCCTCCTTGCCTTCAGC |
| SP5[1] | 72 | TTTTGGGCACGGGCACCTCGCCCTCGTTTT |
| SP8[55] | 73 | CACGGGGCGGGGGTGTTCTGCTGGCGAGCTGC |
| SP8[72] | 74 | TTTTTGTCGGTGGTTTTT |
| SC2[31] | 75 | TGCTCAGGACAGCGGTGAGCGGGCCGTTTGTTCTG |
| | | CTTCGATGTTG |
| SC7[24] | 76 | TTTCTTGATGCCGTTCGTCCAGCTAAGTCGATCTT |
| | | GCTCACCATTTT |
| SC1[2] | 77 | TTGTACTCCAGCCGGTTCACGAACTTGTGACTTGG |
| | | GTG |
| SC8[63] | 78 | TTTCCAGGATGGGCACCACCCCTCCTCGCCGCCCT |
| | | TCA |
| SC2[88] | 79 | TTTTCGCCGATGGG |
| SC6[43] | 80 | AACTTCACTGAAGAAGATGGTGTTGTAG |
| SC1[42] | 81 | CTGCTTCCTGCACGCACGTTGTGGACACGCTCAGG |
| | | GTGTGTTGGGGTCTT |
| SC6[67] | 82 | TCCTCTCGGCGCGTGGCATCAACTTCAGCACGAGG |
| | | G |
| SC4[80] | 83 | TTTTTGCCGTAGGGGTCTTGTTTT |
| SC5[60] | 84 | GGTACGTCGCCTTCTGCTTCAGGATGTTGCCGTCC |
| | | TCCTTTT |
| SC7[1] | 85 | TGTGATCGTGCAGATGGCCCTCGCGGGGTAGCGAA |
| | | GTCGTG |
| SC7[56] | 86 | TGGGCCAGGGCACGGGATGTGGTCCCTCGCCGGCT |
| | | GCGCTCCTG |
| SC2[63] | 87 | CGGGCAGCAGCACGGGGCCGTTTTT |
| SC0[83] | 88 | TTTTTAGTTGCCGTCG |
| SC4[22] | 89 | CTCCTTGCCGGTGGCGCTTCTCCGCCCTCG |
| SC5[32] | 90 | TTTTTGGCGGACTTGAAGGCTGAAGCACTGCACGC |
| | | CGTTTTT |
| SC8[36] | 91 | TTTTGAACTCCAGCAGGACCA |
| SC8[84] | 92 | GCTCGATGTTGTGCCCGTCGGCCA |
| SC3[21] | 93 | TGATATAGTGCCGTCCGGTAGTGGTCGGCGAGCAG |
| | | AGGTAGTGGTTGT |
| SC5[0] | 94 | GACGTAGCCTTCGGGCATTTT |
| SC3[7] | 95 | TTTTTTGCGÅTTT |
| SC4[54] | 96 | TTTTAGGTCAGGGTGGTGGTCAGCTTTTT |
| SC6[83] | 97 | TGGCGGATCTTGAAGTTCACTTT |
| SP8[23] | 98 | AAAAACCACCCCGAACTTGTGGCCGTTTCCGTAGG |
| A4 5′ 3′ | | TAAAA |
| SP4[23] | 99 | AAAATGGTCACGATGTGGTCGGGGTAGCCTTCGGG |
| A4 5′ 3′ | | CAAAA |
| SP4[37] | 100 | AAAAGCCGTAGATGAACTTCACGATGGTCCTGGGT |
| A4 5′ 3′ | | CAAAA |
| SP0[23] | 101 | AAAACTTCACCTCGGCGCGGGTCTTTTAAAA |
| A4 5′ 3′ | | |
| SP6[72] | 102 | AAAATTTTGTCGGTAGTGTTTTAAAA |
| A4 5′ 3′ | | |
| SP3[1] | 103 | AAAATTTTCGTGCTGCTTCAGGGTGGGCCATTTTA |
| A4 5′ 3′ | | AAA |
| SP4[55] | 104 | AAAATCTTCTGCTGATATAGACGTTGTGTTGTACT |
| A4 5′ 3′ | | CAAAA |
| SP7[1] | 105 | AAAATTTTCCGGACACGCTGGTGAACAGCTTTTTA |
| A4 5′ 3′ | | AAA |
| SP4[72] | 106 | AAAATTTTACCTTAGTTCTTTTAAAA |
| A4 5′ 3′ | | |
| SP6[37] | 107 | AAAAAGCTTGACGTCGCCGTCCCGATCGTCGCAGC |
| A4 5′ 3′ | | TCGACCACTTTGCTCAAAAA |
| SP2[23] | 108 | AAAAATGGCGGAGTCGTCCTTGAAGAAGCCCTCGA |
| A4 5′ 3′ | | AAAAA |
| SP2[72] | 109 | AAAATTTTTGTAGGCTGTTTTTAAAA |
| A4 5′ 3′ | | |
| SP9[1] | 110 | AAAATTTTCCTCGCCCTTGCTCACCATGTTGGGGT |
| A4 5′ 3′ | | GGATGGGCAAAA |
| SP0[55] | 111 | AAAATCGATGCGGTTCACCAGGGTGTCGATGGTGC |
| A4 5′ 3′ | | GCTCAGGATGCCCCCTGGAAAAA |
| SP6[55] | 112 | AAAAACGCTGCCTTGTGGCGGATCTTGAGATGCCG |
| A4 5′ 3′ | | TAAAA |
| SP9[41] | 113 | AAAAGGGCGGACTTGGGTGCTCAGGTAGGCAGCAG |
| A4 5′ 3′ | | AAAA |
| SP2[37] | 114 | AAAACGTAGCGGCTGAAGCACGGCCATTGTCTGCA |
| A4 5′ 3′ | | CAAAA |
| SP1[1] | 115 | AAAATTTTTTGTAGTTGCCCTTGAAGAAGTTTTTA |
| A4 5′ 3′ | | AAA |
| SP0[72] | 116 | AAAATTTTGATGCAAGTCTTTTAAAA |
| A4 5′ 3′ | | |
| Non- | 117 | CTCAGTGGACAGCCGTTCTGGAGCGTTGGACGAAA |
| binding | | CT |
| SS | | |

TABLE 5
Construction composition for mRNA-DNA hybrid nanostructures

| Construct | Scaffold mRNA | Staples (DNA) |
|---|---|---|
| mRNA | GFP mRNA | — |
| Linear duplex (LD) | GFP mRNA | LD0[37] to LD0[317] |
| Helical bundle (HB) | GFP mRNA | HBC3[13] to HBC4[107 |
| Square plate (SP) | GFP mRNA | SP8[23] to SP8[72] |
| Cube (CB) | GFP mRNA | SC1[2] to SC6[83] |
| Hairy square plate | GFP mRNA | SP8[23] to SP8[72] |
| (HSP) | | |
| Hairy square plate | GFP mRNA | SP0[23], SP3[1], SP2[23], |
| (HSP)-18 | | SP2[72], SP0[55] |
| Hairy square plate | GFP mRNA | SP0[23] |
| (HSP)-22 | | |
| Non-binding SS | GFP mRNA | Non-binding SS |

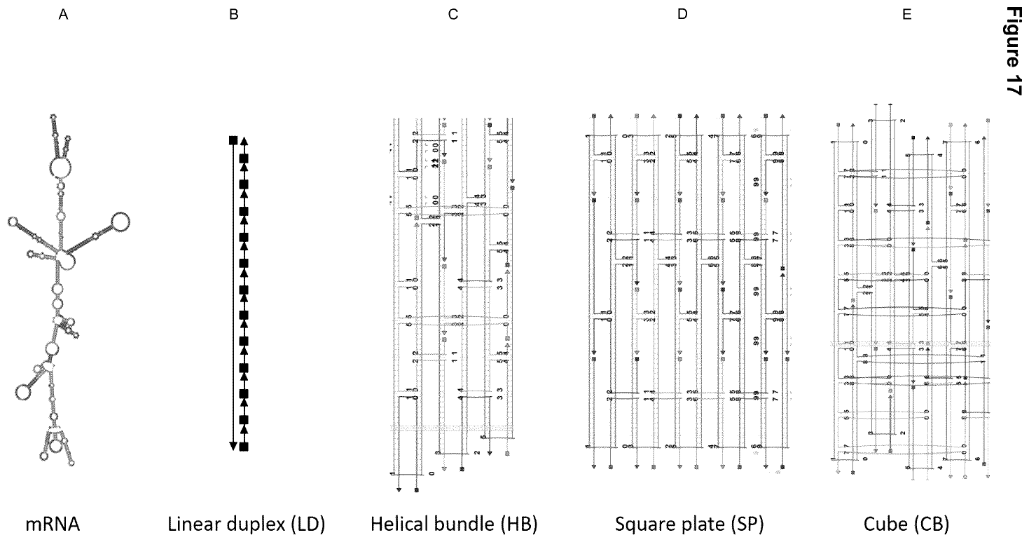

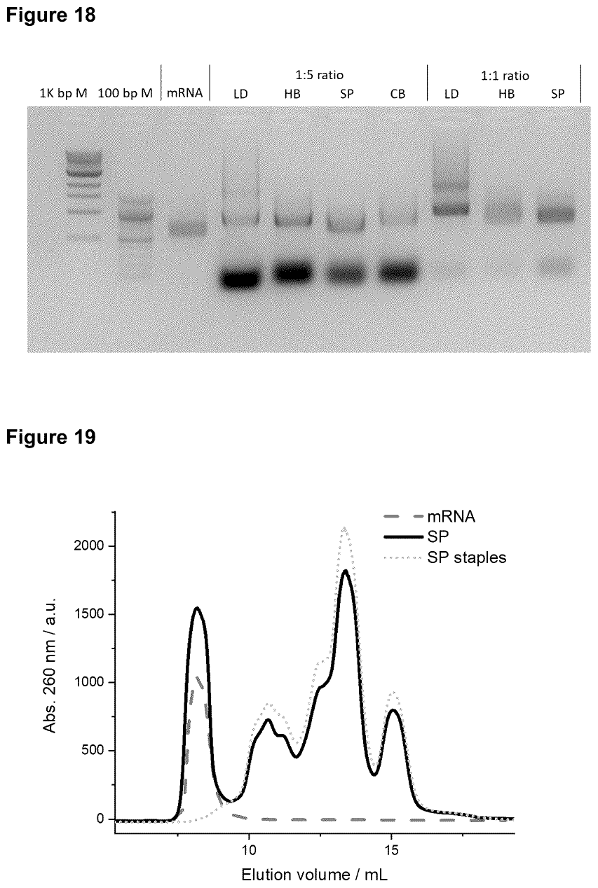

The mRNA-DNA nanostructures were formed via self-assembly. The mixtures of the GFP mRNA and DNA oligonucleotide staple strands at a ratio of either 1:5 or 1:1 were mixed in buffer 1×TAE pH 8.3 supplemented with 300 mM KCl and heated up to 60° C. and cooled down to 4° C. The assembly products were analysed via gel electrophoresis (FIG. 18). All mRNA-DNA nanostructures assembled as indicated by the product band which migrated higher than the mRNA scaffold. The assembly was independent of whether the ratio of scaffold to staples was 1:5 or 1:1. The amount of excess staple strands, indicated by a stronger band, was higher for the 1:5 ratio than for the 1:1 ratio, as expected.

The assembly mix of the mRNA-DNA nanostructures was analysed by size exclusion chromatography (SEC). As illustrated for the assembly mixture of the square plate (FIG. 19), the SEC chromatogram featured peaks at 8 mL, 11.5 mL, 13.5 mL, and 15.5 mL. The first peak at 7.5 mL coincided with the peak of the mRNA scaffold as the mRNA-DNA assembly product could not be resolved by SEC from the mRNA scaffold peak. The other three peaks are from staple strands.

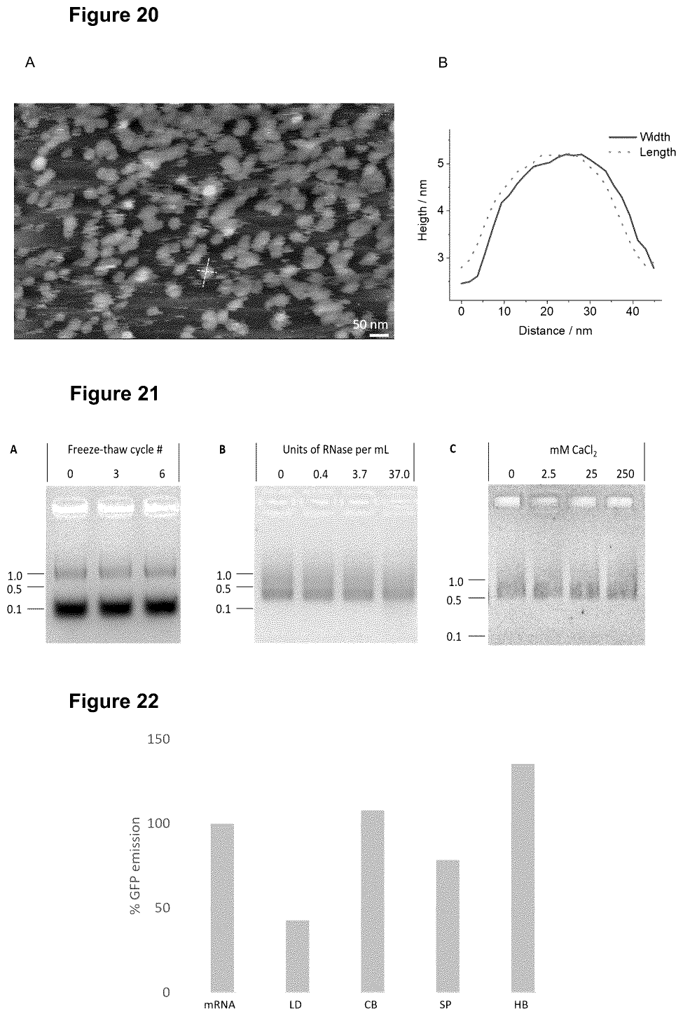

The SEC-purified assembly products were subjected to atomic force microscopy (AFM) to confirm the formation of the nanostructures. As illustrated for the square plate (FIG. 20, left), AFM micrographs featured square planar nanostructures. Their size was 25×25 nm and a depth of around 2 nm within the height profile (FIG. 20, right), in line with the expected nominal dimensions.

The stability of the mRNA-DNA hybrid nanostructures against repeating thawing and freezing as well as nuclease digestion (RNase H) was assessed by gel electrophoresis (FIG. 21).

The capability of the mRNA-DNA hybrid to serve as a substrate for translation was probed by incubation with a cell-free expression mixture featuring ribosomes and loaded tRNAs, among other components. The outcome of successful in vitro expression of the GFP mRNA was determined by fluorescence measurements to detect the level of green fluorescent protein (GFP). FIG. 22 summarises the fluorescent readings equivalent to the expression levels from several mRNA-DNA nanostructures. When compared to pure mRNA, the linear duplex (LD) featured around 50% of the expression level, while the square plate (SP) was at 75%. The cube (CB) had about the same expression level and the helix bundle (HB) was at 130%. The results indicate that all mRNA-DNA nanostructures were able to be successfully translated into active protein, with the cube (CB) and helical bundle (HB) nanostructures demonstrating surprisingly higher levels of expression compared to naked mRNA alone. Different levels of expression for the various mRNA-DNA structures may indicate the steric and/or energetic effects on the ability to read the mRNA sequence by the in vitro translation machinery.

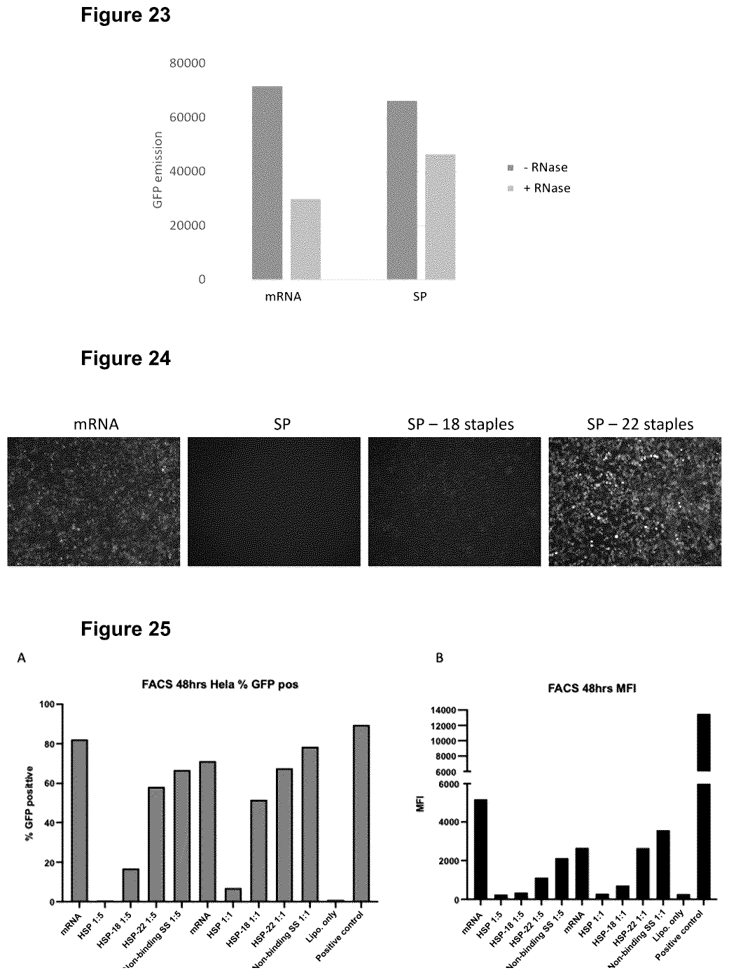

The cell-free expression was also used to probe the stabilising effect of the nanostructure of the mRNA against nuclease digestion. In the assay, the GFP mRNA-DNA square plate (SP) as well as mRNA as control were incubated with or without RNase for 30 mins at 37° C., followed by addition of cell-free in vitro expression reagents for the production of GFP. The results of the GFP fluorescence reading are shown in FIG. 23. The data indicate that incorporating the mRNA into the nanostructure renders the mRNA more nuclease resistant when compared to the naked mRNA.

The cellular uptake and cellular expression of the mRNA-DNA nanostructures was also examined. In these experiments, the square plate with a full set of 23 complementary DNA staple strands, a square plate with 5 staple strands, and a square plate with one staple strand were used (see Table 4 for composition) as well as the mRNA strand. The staples featured additional unpaired nucleotide extensions at the 5′ and 3′ terminus (Table 5) to facilitate the steric accessibility of the ribosome to the mRNA and help the unzipping of the staple strands. Given the unpaired extensions, the square plate structures are abbreviated hairy square plate (HSP) yielding HSP for the structure with all 23 staples, HSP-18 for the one with only 5, and HSP-22 with one staple. The different square plates were assembled either with an excess of staple DNA strands to the mRNA scaffold of 5:1 molar ratio, or at a ratio of 1:1. The constructs were hence defined as, for example, HSP(1:5) or HSP-22(1:1). As control, GFP mRNA was also incubated with single stranded DNA staples not matching the mRNA sequence (non-binding SS). After assembly, the mRNA/DNA constructs and the DNA-free mRNA control were mixed with lipofectamine reagent and then used to transfect HeLa cells.

The cells were grown for 48 h and imaged first by fluorescence microscopy. Microscopic images of the cells are shown in FIG. 24. As shown by the fluorescence signal, the cells were able to take up the HSP structures and read the mRNA from the mRNA-DNA hybrid structures by unravelling the mRNA component from the nanostructures during translation.

The cells were also analysed via fluorescence activated cell sorting (FACS) to determine the % of cells that express GFP as well as from the positive cells the mean fluorescence intensity (MFI) which is equivalent to the level of GFP expression minus any degradation. The FACS data on the percentage of expressing cells and MFI are summarised in FIGS. 25A and 25B, respectively. The data show that transfection of GFP mRNA plus water resulted in positive GFP expression in ˜80% of cells (FIG. 25A). Complexing of the mRNA with different staples also resulted in detectable GFP cellular expression, with the exception of HSP(1:5) (FIG. 25A). For several constructs, 60-70% of cells became GFP positive (FIG. 25A). Two trends were noted. First, decreasing the number of staples increased the expression level from HSP over HSP(−18) to HSP(−22) (FIG. 25A). As additional trend, a lower ratio of staple strands (1:1) vs (1:5) led to higher expression levels (FIG. 25A). The ability to tune the expression level of mRNA in cells via the number of staples present is surprising and represents an additional mechanism for control of expression and stability in the delivered mRNA.

In conclusion, these results show that RDH nanostructures significantly improve the stability of an mRNA in the presence of RNAses, DNAses and through thermal cycles. In addition, the mRNA retains the ability to be expressed at significant levels. Finally, a human cancer cell line is able to take up the delivered nanostructures and expresses the gene product at significant detectable levels.

These results may also be combined with the those obtained for Example 1, which show that the use of membrane binding moieties can affect the selectivity of the nanostructures for target recipient cells. Hence, the present invention provides a novel platform for coding nucleic acid, especially mRNA, delivery to cells which has much enhanced stability and durability under environmental and physiological conditions.

Materials and Methods:

Reagents: All DNA oligonucleotides were purchased from IDT DNA technologies (Coralville, IA, USA) on a 100 nmol scale, desalted. CleanCap EGFP mRNA (5 moU) was procured from Trilink Biotechnologies (UK) on a 1 mg scale. All other reagents were purchased from Sigma-Merck (UK) unless stated.

CaDNAno. The hybrid mRNA-DNA origami constructs were designed using CaDNAno (https://cadnano.org/). The GFP mRNA sequence was used as the scaffold strand. Mis-matches were included in the origami designs to prevent blunt-end stacking interactions.

Mixing and folding GFP mRNA constructs. To fold the constructs, GFP mRNA (46.5 μL, 4300 nM), pooled staples strands (80 μL, 12.5 NM) and 10×TAE 0.3 mM KCl pH 8.3 (100 μL) were added to deionised water (773.5 μL). The constructs were annealed by heating to 65° C. for 2 mins then cooling to 4° C. at a rate of 1° C. per minute. For the mRNA only construct the pooled staples strands were replaced with deionised water.

Agarose gel electrophoresis. Ultra-pure agarose (2.1 g) and 1×TAE pH 8.3 (150 mL) were added to a conical flask (250 mL). The agarose was dissolved using a microwave, the evaporated water replenished, then poured into a gel cast along with comb and ethidium bromide solution (5 μL, 10 mg per mL). The set gel was transferred to the gel tank and submerged in running buffer (400 mL, 1×TAE) at 4° C. The samples were loaded. Purple DNA loading dye (5 μL, 6×) (New England Biolabs, UK) was added to the stated sample (20 μL). A fraction of this solution (10 μL) was loaded onto the gel. 1000 bp and 100 bp markers were used as a reference (New England Biolabs, UK). The gel was run at 60 volts for 1 hr at 4° C. The gel was imaged using a gel reader (200 Azure Biosystems, UK).

Size exclusion chromatography. Where stated, SEC was conducted to remove excess staple strands using an Akta FPLC (GE Healthcare, UK) and S200 column (GE Healthcare, UK). The stated constructs were injected (0.9 mL, 4300 nM) onto the column at a flow rate of 0.5 mL per min at room temperature, monitoring the absorbance at 280, 260 and 345 nm. The samples were collected in 0.25 mL fractions, four fractions which eluted at 8 mL were combined and stored in the fridge until required.

Atomic force microscopy. AFM analysis was performed on SEC-purified SP using a Multimode 8 (Nanoscope, Bruker AXS, US) and MSNL-10 cantilevers (Bruker AFM Probes, US). The DNA nanostructure (2200 nM, 2 μL) was deposited on freshly cleaved mica and allowed to adhere for 5 min. The sample was then supplemented with 1×TAE 14 mM MgCl₂ (80 μL). The images were collected using voltage engage set points between 10-40 mV, scanning at 4 Hz, scan area 1 μm×1 μm at 512 pixels per line, feedback gain of 20, and z-height limit of 1.5 μm. The image was processed using Gwyddion software (http://gwyddion.net/).

RNase stability assay. RNase H (2 μL, 5000 units per mL) (New England Biolabs, UK) and RNase H buffer (2.5 μL, 10×) (New England Biolabs, UK) was added to SEC-purified SP (25 μL, 2200 nM). A serial dilution was performed by diluting the sample (2.5 μL) into fresh construct (25 μL, 2200 nM) three times. The samples were incubated at 37° C. for 4 hr. Next, 1.5% agarose gel electrophoretic analysis was performed following the above protocol.

Freeze-thawing stability assay. SP (25 μL, 4300 nM) was frozen at −80° C. for 5 mins, then incubated at 37° C. for 5 mins. The freeze-thaw cycles were performed up to 10 times. Next, 1.5% agarose gel electrophoretic analysis was performed following the above protocol.

Divalent metal stability assay. Magnesium chloride (0.63 μL 1000 mM) and calcium chloride (0.25 μL 1000 mM) was added to SEC-purified SP (25 μL, 2200 nM). A serial dilution was performed by diluting the sample (2.5 μL) into fresh construct (25 μL, 2200 nM) three times, then incubated for 4 hr at 37° C. Next, 1.5% agarose gel electrophoretic analysis was performed following the above protocol.

Cell-free protein expression. The stated constructs (2.5 μL, 4300 nM, folded in a scaffold to staple ratio of 1:5) were added to protein synthesis buffer (25 μL) (New England Biolabs, UK) and cell extract (12.5 μL) (New England Biolabs, UK) in a PCR tube (100 μL) and mixed at 37° C. for 6 hr. The amount of GFP produced was quantified using a fluorescence spectrophotometer (Cary Eclipse, Agilent, UK). Excitation wavelength 495 nm, emission 515 nm, PMT voltage 600. For the RNase stability assay, RNase H (2 μL, 5000 units per mL) (New England Biolabs, UK) and RNase H buffer (2.5 μL, 10×) (New England Biolabs, UK) was added to the stated GFP mRNA constructs (2.5 μL, 4300 nM, folded in a scaffold to staple ratio of 1:5) and incubated for 30 mins at 37° C. Then the above procedure for cell-free protein expression was followed.

Cell culture and flow cytometry. HeLa cells were plated at 80% confluency 1 day prior to infection. On the day of transfection, cells were cultured in low serum Opti-MEM media (GIBCO). GFP mRNA or GFP mRNA plus staples were incubated with lipofectamine reagent as described by manufacturer (Invitrogen) to form lipid:nucleic acid complexes. After 15 minutes, these complexes were added to cells and then after 4 hr the media was replaced with DMEM (GIBCO) supplemented with 10% fetal bovine serum (GIBCO). At 48 hr post transfection, cells were trypsinised to remove them from the plates, pelleted at 300 g for 5 minutes and then resuspended in 100 ul PBS. Cells were then analysed by flow cytometry for GFP expression using a Becton Analyser. Gating was performed on untransfected cells to set the levels of background cell autofluorescence in the FL1 channel.

Although particular embodiments of the invention have been disclosed herein in detail, this has been done by way of example and for the purposes of illustration only. The aforementioned embodiments are not intended to be limiting with respect to the scope of the appended claims, which follow. The choice of nucleic acid starting material, the clone of interest, or type of library used is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

1. A nucleic acid nanostructure comprising:
a first single stranded nucleic acid sequence that defines a scaffold sequence, wherein the scaffold sequence comprises at least one open reading frame that encodes a first gene product; and
a plurality of single stranded nucleic acid sequences that define a plurality of staple sequences, wherein the plurality of staple sequences are capable of hybridising with one or more regions of the scaffold sequence in order to induce the formation of a geometrically predefined higher order structure.

2. The nucleic acid nanostructure of claim 1, further comprising at least one membrane binding moiety, wherein the membrane binding moiety is configured to associate with a cell membrane.

3. The nucleic acid nanostructure of claim 1 or 2, wherein the scaffold sequence is comprised of RNA or an analogue thereof, optionally wherein the RNA is a messenger RNA (mRNA).

4. The nucleic acid nanostructure of claim 1, wherein the plurality of staple sequences are comprised of DNA or a DNA analogue.

5. The nucleic acid nanostructure of claim 1, wherein the nanostructure comprises at least a second single stranded nucleic acid sequence that defines a second scaffold sequence, and wherein the plurality of staple sequences are capable of hybridising with one or more regions of both the first and the second scaffold sequences in order to induce the formation of a geometrically predefined higher order structure.

6. The nucleic acid nanostructure of claim 5, wherein the second scaffold sequence comprises at least one open reading frame that encodes a second gene product, wherein the second gene product may be the same or different to the first gene product.

7. The nucleic acid nanostructure of claim 5, wherein the nanostructure comprises at least a third single stranded nucleic acid sequence that defines a third scaffold sequence, and wherein the plurality of staple sequences are capable of hybridising with one or more regions of the first, second and the third scaffold sequences in order to induce the formation of a geometrically predefined higher order structure.

8. The nucleic acid nanostructure of claim 7, wherein the third scaffold sequence comprises at least one open reading frame that encodes a third gene product, wherein the third gene product may be the same or different to the first and/or second gene products.

9. The nucleic acid nanostructure of claim 1, wherein the gene product is selected from one or more the group consisting of: an antigen; an immunomodulator; an antibody or a fragment thereof; an aptamer; a cytokine; an enzyme; and a reporter protein.

10. The nucleic acid nanostructure of claim 9, wherein the antigen comprises all or part of any of the group consisting of:
i. a SARS-CoV-2 spike protein, or a receptor binding domain (RBD) and any variant thereof;
ii. a human cytomegalovirus antigen—such as glycoprotein B, PP65 and/or IE1;
iii. a hepatitis C (HCV) antigen;
iv. a human immunodeficiency virus (HIV) antigen;
v. a respiratory syncytial virus (RSV) antigen;
vi. an Ebola virus antigen;
vii. a tuberculosis antigen;
viii. a malaria antigen;
ix. a tumoral neoantigen;
x. a tumor-associated antigen; and
xi. an influenza virus antigen.

11. (canceled)

12. The nucleic acid nanostructure of claim 2, wherein membrane binding moiety comprises a hydrophobic moiety, wherein the hydrophobic moiety comprises a non-polar or low polarity aliphatic, aliphatic-aromatic or aromatic chain, or wherein the hydrophobic moiety is selected from the group consisting of: a long chain carbocyclic molecule; a hydrophobic polymer or block co-polymers; and a lipid.

13. (canceled)

14. The nucleic acid nanostructure of claim 12, wherein the lipid is selected from one or more of the group consisting of: cholesterol; derivatives of cholesterol; phytosterol; ergosterol; bile acid; alkylated phenols (including methylated phenols and tocopherols); flavones (including flavanone containing compounds such as 6-hydroxyflavone); saturated and unsaturated fatty acids (including derivatives such as lauric, oleic, linoleic and palmitic acids); and synthetic lipid molecules (including dodecyl-beta-D-glucoside).

15. The nucleic acid nanostructure of claim 1, wherein the nanostructure has a maximum dimension of less than around 100 nm.

16. An RNA-DNA hybrid (RDH) nucleic acid nanostructure comprising
a first single stranded nucleic acid sequence that defines a scaffold sequence, wherein the scaffold sequence is comprised of RNA and includes at least one open reading frame that encodes a first gene product;
a plurality of single stranded DNA sequences that define a plurality of staple sequences, wherein the plurality of staple sequences are capable of hybridising with one or more regions of the scaffold sequence in order to induce the formation of a geometrically predefined higher order structure within the RDH; and
at least one hydrophobic membrane binding moiety, wherein the membrane binding moiety is configured to associate with a cell membrane.

17. The RDH nanostructure of claim 16, wherein the RNA is a messenger RNA (mRNA).

18. The RDH nanostructure of claim 16, wherein the nanostructure comprises at least a second single stranded nucleic acid sequence that defines a second scaffold sequence, and wherein the plurality of staple sequences are capable of hybridising with one or more regions of both the first and the second scaffold sequences in order to induce the formation of a geometrically predefined higher order structure.

19. The RDH nanostructure of claim 18, wherein the second scaffold sequence is comprised of RNA and further comprises at least one open reading frame that encodes a second gene product, wherein the second gene product may be the same or different to the first gene product.

20. The RDH nanostructure of claim 18, wherein the nanostructure comprises at least a third single stranded nucleic acid sequence that defines a third scaffold sequence, and wherein the plurality of staple sequences are capable of hybridising with one or more regions of the first, second and the third scaffold sequences in order to induce the formation of a geometrically predefined higher order structure.

21. The RDH nanostructure of claim 20, wherein the third scaffold sequence is comprised of RNA and further comprises at least one open reading frame that encodes a third gene product, wherein the third gene product may be the same or different to the first and/or second gene products.

22. The RDH nanostructure of claim 16, wherein the gene product is selected from one or more the group consisting of: an antigen; an immunomodulator; an antibody or a fragment thereof; an aptamer; a cytokine; and a reporter protein.

23. The RDH nanostructure of claim 22, wherein the antigen comprises all or part of any of the group consisting of:
i. a SARS-CoV-2 spike protein, or a receptor binding domain (RBD) and any variant thereof;
ii. a human cytomegalovirus antigen—such as glycoprotein B, PP65 and/or IE1;
iii. a hepatitis C (HCV) antigen;
iv. a human immunodeficiency virus (HIV) antigen;
v. a respiratory syncytial virus (RSV) antigen;
vi. an Ebola virus antigen;
vii. a tuberculosis antigen;
viii. a malaria antigen;
ix. a tumoral neoantigen;
x. a tumor-associated antigen; and
xi. an influenza virus antigen.

24-25. (canceled)

26. The RDH nanostructure of claim 16, wherein the hydrophobic membrane binding moiety comprises a non-polar or low polarity aliphatic, aliphatic-aromatic or aromatic chain, or wherein the hydrophobic moiety is selected from the group consisting of: a long chain carbocyclic molecule; a hydrophobic polymer or block co-polymers; and a lipid.

27. The RDH nanostructure of claim 26, wherein the lipid is selected from one or more of the group consisting of: cholesterol; derivatives of cholesterol; phytosterol; ergosterol; bile acid; alkylated phenols (including methylated phenols and tocopherols); flavones (including flavanone containing compounds such as 6-hydroxyflavone); saturated and unsaturated fatty acids (including derivatives such as lauric, oleic, linoleic and palmitic acids); and synthetic lipid molecules (including dodecyl-beta-D-glucoside).

28. A pharmaceutical composition comprising a nanostructure of claim 1 in combination with a suitable excipient.

29-35. (canceled)

36. A method of treating a subject in need thereof, comprising administering to the subject a pharmaceutical composition of claim 28.

37-43. (canceled)

44. A pharmaceutical composition comprising an RDH nanostructure of claim 16 in combination with a suitable excipient.
